SEQUENCE LISTING (4 sequences)

SEQ ID NO: 1 - DNA, 1236 bp, Homo sapiens
tagttcaagacaacagagacaaagctaagatgaggaagttctgtacagtttaggaaatagaggctttcaaagataattcgcagtgatgtgaaactggcctcccaagccctgataacaacatggccaacgccctggccagcgccacttgcgagcgctgcaagggcggctttgcgcccgctgagaagatcgtgaacagtaatggggagctgtaccatgagcagtgtttcgtgtgcgctcagtgcttccagcagttcccagaaggactcttctatgagtttgaaggaagaaagtactgtgaacatgactttcagatgctctttgccccttgctgtcatcagtgtggtgaattcatcattggccgagttatcaaagccatgaataacagctggcatccggagtgcttccgctgtgacctctgccaggaagttctggcagatatcgggtttgtcaagaatgctgggagacacctgtgtcgcccctgtcataatcgtgagaaagccagaggccttgggaaatacatctgccagaaatgccatgctatcatcgatgagcagcctctgatattcaagaacgacccctaccatccagaccatttcaactgcgccaactgcgggaaggagctgactgccgatgcacgggagctgaaaggggagctatactgcctcccatgccatgataaaatgggggtccccatctgtggtgcttgccgacggcccatcgaagggcgcgtggtgaacgctatgggcaagcagtggcatgtggagcattttgtttgtgccaagtgtgagaaaccctttcttggacatcgccattatgagaggaaaggcctggcatattgtgaaactcactataaccagctatttggtgatgtttgcttccactgcaatcgtgttatagaaggtgatgtggtctctgctcttaataaggcctggtgcgtgaactgctttgcctgttctacctgcaacactaaattaacactcaagaataagtttgtggagtttgacatgaagccagtctgtaagaagtgctatgagaaatttccattggagctgaagaaaagacttaagaaactagctgagaccttaggaaggaaataagttcctttattttttcttttctatgcaagataagagattaccaacattacttgtcttgatctacccatatttaaagctatatctcaaagcagttgagagaagaggacctatatgaatggttttatgtcatttttttaaa

SEQ ID NO: 2 - protein, 325 aa, Homo sapiens
MANALASATCERCKGGFAPAEKIVNSNGELYHEQCFVCAQCFQQFPEGLFYEFEGRKYCEHDFQMLFAPCCHQCGEFIIGRVIKAMNNSWHPECFRCDLCQEVLADIGFVKNAGRHLCRPCHNREKARGLGKYICQKCHAIIDEQPLIFKNDPYHPDHFNCANCGKELTADARELKGELYCLPCHDKMGVPICGACRRPIEGRVVNAMGKQWHVEHFVCAKCEKPFLGHRHYERKGLAYCETHYNQLFGDVCFHCNRVIEGDVVSALNKAWCVNCFACSTCNTKLTLKNKFVEFDMKPVCKKCYEKFPLELKKRLKKLAETLGRK

SEQ ID NO: 3 - DNA, 1026 bp, Homo sapiens
atgacgggaagcaatatgtcggacgccttggccaacgccgtgtgccagcgctgccaggcccgcttctcccccgccgagcgcattgtcaacagcaatggggagctgtaccatgagcactgcttcgtgtgtgcccagtgcttccggcccttccccgaggggctcttctatgagtttgaaggccggaagtactgcgaacacgacttccaaatgctgtttgctccgtgctgtggatcctgcggtgagttcatcattggccgcgtcatcaaggccatgaacaacaactggcacccgggctgcttccgctgcgagctgtgtgatgtggagctggctgacctgggctttgtgaagaatgccggcaggcatctctgccggccttgccacaaccgtgagaaggccaaaggcctgggcaagtacatctgccagcggtgccacctggtcatcgacgagcagcccctcatgttcaggagcgacgcctaccaccctgaccacttcaactgcacccactgtgggaaggagctgacagccgaggcccgcgagctgaagggtgagctctactgcctgccctgccatgacaagatgggcgtccccatctgcggggcctgccgccggcccatcgagggccgagtggtcaacgcgctgggcaagcagtggcacgtggagcactttgtctgtgccaagtgtgagaagccattcctggggcaccggcactatgagaagaagggcctggcctactgcgagactcactacaaccagctcttcggggacgtctgctacaactgcagccatgtgattgaaggcgatgtggtgtcggccctcaacaaggcctggtgtgtgagctgcttctcctgctccacctgcaacagcaagctcaccctgaaggacaagtttgtggagttcgacatgaagcccgtgtgtaagaggtgctacgagaagttcccgctggagctgaagaagcggctgaagaagctgtcggagctgacctcccgcaaggcccagcccaaggccacagacctcaactctgcctga

SEQ ID NO: 4 - protein, 476 aa, Homo sapiens
MANALASATCERCKGGFAPAEKIVNSNGELYHEQCFVCAQCFQQFPEGLFYEFEGRKYCEHDFQMLFAPCCHQCGEFIIGRVIKAMNNSWHPECFRCDLCQEVLADIGFVKNAGRHLCRPCHNREKARGLGKYICMTGSNMSDALANAVCQRCQARFSPAERIVNSNGELYHEHCFVCAQCFRPFPEGLFYEFEGRKYCEHDFQMLFAPCCGSCGEFIIGRVIKAMNNNWHPGCFRCELCDVELADLGFVKNAGRHLCRPCHNREKAKGLGKYICQRCHLVIDEQPLMFRSDAYHPDHFNCTHCGKELTAEARELKGELYCLPCHDKMGVPICGACRRPIEGRVVNALGKQWHVEHFVCAKCEKPFLGHRHYEKKGLAYCETHYNQLFGDVCYNCSHVIEGDVVSALNKAWCVSCFSCSTCNSKLTLKDKFVEFDMKPVCKRCYEKFPLELKKRLKKLSELTSRKAQPKATDLNSA

(19) United States
(12) Patent Application Publication
(10) Pub. No.: US 2015/0147766 A1
(43) Pub. Date: May 28, 2015

(54) METHOD FOR DETERMINING DISEASE SEVERITY IN TAUOPATHY-RELATED NEURODEGENERATIVE DISORDERS

(71) Applicant: TEMPLE UNIVERSITY - OF THE COMMONWEALTH SYSTEM OF HIGHER EDUCATION, PHILADELPHIA, PA (US)

(72) Inventors: Teresa Dianne Langford, Bala Cynwyd, PA (US); Salim Merali, Bryn Mawr, PA (US)

(21) Appl. No.: 14/403,876

(22) PCT Filed: May 23, 2013
(86) PCT No.: PCT/US13/42442

§ 371 Date: Nov. 25, 2014

Related U.S. Application Data
(60) Provisional application No. 61/652,510, filed on May 29, 2012.

(51) Int. Cl. G01N 33/68; C12Q 1/37

(52) U.S. Cl. CPC G01N 33/6896; C12Q 1/37; G01N 2333/47; G01N 2800/2821; G01N 2800/2814; G01N 2560/00; G01N 2333/96441; G01N 2800/56
USPC 435/7.92; 436/501; 435/24; 435/7.1

(57) ABSTRACT

A method for determining disease severity in a subject afflicted with a tauopathy-related neurodegenerative disease comprises detecting the level of a PINCH protein or isoform thereof in a test sample comprising brain tissue or cerebrospinal fluid. The PINCH protein level in the test sample indicates the relative severity of the tauopathy-related neurodegenerative disease afflicting the subject.

### CROSS-REFERENCE TO RELATED APPLICATION

The benefit of the filing date of U.S. Provisional Patent Application No. 61/652,510, filed May 29, 2012, is hereby claimed. The entire disclosure of the aforesaid application is incorporated herein by reference.

### REFERENCE TO GOVERNMENT GRANT

This invention was made with government support under grant no. MH085602 awarded by the National Institutes of Health. The government has certain rights in this invention.

### SEQUENCE LISTING

The instant application contains a Sequence Listing which has been submitted in ASCII format via EFS-Web and is hereby incorporated by reference in its entirety. Said ASCII copy, created on May 22, 2013, is named 035926_{—}0458_{—}00_WO_SL.txt and is 10,548 bytes in size.

### FIELD OF THE INVENTION

The invention relates to methods for determining disease severity and disease progression, particularly in neurodegenerative disorders.

### BACKGROUND OF THE INVENTION

Tauopathies are a class of neurodegenerative diseases associated with the pathological aggregation of tau protein in the brain. In one such tauopathy, Alzheimer's disease (AD), tau protein is deposited within neurons in the form of neurofibrillary tangles (NFTs). Tangles are formed by hyperphosphorylation of tau, causing it to aggregate in an insoluble form. Hyperphosphorylated tau (hp-Tau) detaches from microtubules and can form paired helical filaments (PHF) and tangles leading to neuronal dysfunction. In healthy ageing, aberrant proteins such as hp-Tau may be cleared from the cell by the heat shock response (HSR) machinery.

The HSR includes surveillance of proteins post-translationally and after cellular insult or stress. These stressors include diverse events such as traumatic brain injury, (TBI), chronic traumatic encephalopathy (CTE) injury, epileptic seizures, Alzheimer's disease (AD), HIV encephalitis (HIVE), and frontotemporal dementia (FTD), and can manifest, in part as accumulation of hp-Tau. Proteins that cannot be repaired may be targeted to the ubiquitin-proteasome system (UPS) for degradation. The HSR works with the UPS for the recognition and clearance of abnormal/aberrant proteins through the binding of a series of chaperone proteins and attachment of ubiquitin molecules to the client protein.

In the case of hp-Tau, the HSR complex sorts aberrant Tau for either repair or degradation. However, if the cellular machinery fails to clear abnormal proteins, accumulation of aberrant proteins can occur. Accumulation of hp-Tau accounts for more than 20 neuropathological diseases including AD and HIVE.

Growing evidence points to significant overlap between mechanisms involved in HIV-associated neurocognitive disorders (HAND) and age-related neurodegenerative diseases, such as AD. HIV+ individuals diagnosed decades ago are beginning to face age-associated CNS changes. Combined with infection and long-term exposure to combination anti-retroviral therapy, age-related neurodegeneration is exacerbated.

Tau is ubiquitously expressed in the brain, and assembles and stabilizes microtubules in neuronal axons. Normally, the HSR complex sorts aberrant hp-Tau for either repair or degradation, as described above. Upon hyperphosphorylation, Tau dissociates from microtubules and may be redistributed to the cell body and dendrites where it accumulates and forms fibrillary deposits consisting of PHF to form tangles. Neurofibrillary tangles are composed in part of ubiquitinated hp-Tau and recent studies report that both the proteasomal and autophagosomal pathways are involved in hp-Tau degradation, but controversy exists regarding the preferential degradation of specific forms of Tau (Dickey, et al. (2007) J Clin Invest 117(3), 648-658; Dolan and Johnson,. J Biol Chem 285(29), 21978-21987).

PINCH is a highly conserved protein composed of 5 double zinc finger domains and has no reported catalytic activity. PINCH is a key component in the formation of multi-protein complexes, and facilitates cell spreading, migration and survival. One of PINCH's most studied binding partners, integrin linked kinase (ILK), has been shown to interfere with GSK3-β-mediated Tau phosphorylation and in some systems, ILK's activity is dependent on its binding to PINCH (Ishii et al., (2003) J Biol Chem 278(29), 26970-26975).

PINCH (now PINCH-1) (GenBank accession # U09284) consists of five LIM domains, each with unique sequences, and lacks a catalytic domain. In the first zinc finger of PINCH's LIM domains three and four, C2H2 is present rather than C2HC. Also, in the fifth LIM domain, a C4HC is substituted for C2HC, potentially altering the three-dimensional structure of the domain and therefore its binding specificity.

The nucleotide (SEQ ID NO:1) and amino acid (SEQ ID NO:2) sequences of human PINCH-1 are:

(SEQ ID NO: 1)
tagttcaaga caacagagac aaagctaaga tgaggaagtt
ctgtacagtt taggaaatag aggctttcaa agataattcg
cagtgatgtg aaactggcct cccaagccct gataacaaca
tggccaacgc cctggccagc gccacttgcg agcgctgcaa
gggcggcttt gcgcccgctg agaagatcgt gaacagtaat
ggggagctgt accatgagca gtgtttcgtg tgcgctcagt
gcttccagca gttcccagaa ggactcttct atgagtttga
aggaagaaag tactgtgaac atgactttca gatgctcttt
gccccttgct gtcatcagtg tggtgaattc atcattggcc
gagttatcaa agccatgaat aacagctggc atccggagtg
cttccgctgt gacctctgcc aggaagttct ggcagatatc
gggtttgtca agaatgctgg gagacacctg tgtcgcccct
gtcataatcg tgagaaagcc agaggccttg ggaaatacat
ctgccagaaa tgccatgcta tcatcgatga gcagcctctg
atattcaaga acgaccccta ccatccagac catttcaact
gcgccaactg cgggaaggag ctgactgccg atgcacggga
gctgaaaggg gagctatact gcctcccatg ccatgataaa
atgggggtcc ccatctgtgg tgcttgccga cggcccatcg
aagggcgcgt ggtgaacgct atgggcaagc agtggcatgt
ggagcatttt gtttgtgcca agtgtgagaa accctttctt
ggacatcgcc attatgagag gaaaggcctg gcatattgtg
aaactcacta taaccagcta tttggtgatg tttgcttcca
ctgcaatcgt gttatagaag gtgatgtggt ctctgctctt
aataaggcct ggtgcgtgaa ctgctttgcc tgttctacct
gcaacactaa attaacactc aagaataagt ttgtggagtt
tgacatgaag ccagtctgta agaagtgcta tgagaaattt
ccattggagc tgaagaaaag acttaagaaa ctagctgaga
ccttaggaag gaaataagtt cctttatttt ttcttttcta
tgcaagataa gagattacca acattacttg tcttgatcta
cccatattta aagctatatc tcaaagcagt tgagagaaga
ggacctatat gaatggtttt atgtcatttt tttaaa.
(SEQ ID NO: 2)
Met Ala Asn Ala Leu Ala Ser Ala Thr Cys Glu
Arg Cys Lys Gly Gly Phe Ala Pro Ala Glu Lys
Ile Val Asn Ser Asn Gly Glu Leu Tyr His Glu
Gln Cys Phe Val Cys Ala Gln Cys Phe Gln Gln
Phe Pro Glu Gly Leu Phe Tyr Glu Phe Glu Gly
Arg Lys Tyr Cys Glu His Asp Phe Gln Met Leu
Phe Ala Pro Cys Cys His Gln Cys Gly Glu Phe
Ile Ile Gly Arg Val Ile Lys Ala Met Asn Asn
Ser Trp His Pro Glu Cys Phe Arg Cys Asp Leu
Cys Gln Glu Val Leu Ala Asp Ile Gly Phe Val
Lys Asn Ala Gly Arg His Leu Cys Arg Pro Cys
His Asn Arg Glu Lys Ala Arg Gly Leu Gly Lys
Tyr Ile Cys Gln Lys Cys His Ala Ile Ile Asp
Glu Gln Pro Leu Ile Phe Lys Asn Asp Pro Tyr
His Pro Asp His Phe Asn Cys Ala Asn Cys Gly
Lys Glu Leu Thr Ala Asp Ala Arg Glu Leu Lys
Gly Glu Leu Tyr Cys Leu Pro Cys His Asp Lys
Met Gly Val Pro Ile Cys Gly Ala Cys Arg Arg
Pro Ile Glu Gly Arg Val Val Asn Ala Met Gly
Lys Gln Trp His Val Glu His Phe Val Cys Ala
Lys Cys Glu Lys Pro Phe Leu Gly His Arg His
Tyr Glu Arg Lys Gly Leu Ala Tyr Cys Glu Thr
His Tyr Asn Gln Leu Phe Gly Asp Val Cys Phe
His Cys Asn Arg Val Ile Glu Gly Asp Val Val
Ser Ala Leu Asn Lys Ala Trp Cys Val Asn Cys
Phe Ala Cys Ser Thr Cys Asn Thr Lys Leu Thr
Leu Lys Asn Lys Phe Val Glu Phe Asp Met Lys
Pro Val Cys Lys Lys Cys Tyr Glu Lys Phe Pro
Leu Glu Leu Lys Lys Arg Leu Lys Lys Leu Ala
Glu Thr Leu Gly Arg Lys.

Following the discovery of PINCH (PINCH-1, GenBank accession # U09284; SEG ID NO:1 and NO:2)), a related protein PINCH-2 (GenBank accession # AF484961.1), was characterized. PINCH-1 and PINCH-2 share approximately 82% amino acid sequence homology, but are encoded by separate genes. Although PINCH-1 and -2 are co-expressed, they appear to be functionally distinct, with PINCH-2 potentially mediating the PINCH-1/integrin linked kinase (ILK) interaction. Mammals contain both PINCH-1 and PINCH-2 (Chiswell et al., (2010) Journal of structural biology 170(1), 157-163). In PINCH-1 depletion studies, PINCH-2 could not compensate for cell spreading and cell survival.

The nucleotide (SEQ ID NO:3) and amino acid (SEQ ID NO:4) sequences of human PINCH-2 (GenBank accession # AF484961.1) are:

(SEQ ID NO: 3)
atgacgggaa gcaatatgtc ggacgccttg gccaacgccg
tgtgccagcg ctgccaggcc cgcttctccc ccgccgagcg
cattgtcaac agcaatgggg agctgtacca tgagcactgc
ttcgtgtgtg cccagtgctt ccggcccttc cccgaggggc
tcttctatga gtttgaaggc cggaagtact gcgaacacga
cttccaaatg ctgtttgctc cgtgctgtgg atcctgcggt
gagttcatca ttggccgcgt catcaaggcc atgaacaaca
actggcaccc gggctgcttc cgctgcgagc tgtgtgatgt
ggagctggct gacctgggct ttgtgaagaa tgccggcagg
catctctgcc ggccttgcca caaccgtgag aaggccaaag
gcctgggcaa gtacatctgc cagcggtgcc acctggtcat
cgacgagcag cccctcatgt tcaggagcga cgcctaccac
cctgaccact tcaactgcac ccactgtggg aaggagctga
cagccgaggc ccgcgagctg aagggtgagc tctactgcct
gccctgccat gacaagatgg gcgtccccat ctgcggggcc
tgccgccggc ccatcgaggg ccgagtggtc aacgcgctgg
gcaagcagtg gcacgtggag cactttgtct gtgccaagtg
tgagaagcca ttcctggggc accggcacta tgagaagaag
ggcctggcct actgcgagac tcactacaac cagctcttcg
gggacgtctg ctacaactgc agccatgtga ttgaaggcga
tgtggtgtcg gccctcaaca aggcctggtg tgtgagctgc
ttctcctgct ccacctgcaa cagcaagctc accctgaagg
acaagtttgt ggagttcgac atgaagcccg tgtgtaagag
gtgctacgag aagttcccgc tggagctgaa gaagcggctg
aagaagctgt cggagctgac ctcccgcaag gcccagccca
aggccacaga cctcaactct gcctga,
(SEQ ID NO: 4)
Met Ala Asn Ala Leu Ala Ser Ala Thr Cys Glu
Arg Cys Lys Gly Gly Phe Ala Pro Ala Glu Lys
Ile Val Asn Ser Asn Gly Glu Leu Tyr His Glu
Gln Cys Phe Val Cys Ala Gln Cys Phe Gln Gln
Phe Pro Glu Gly Leu Phe Tyr Glu Phe Glu Gly
Arg Lys Tyr Cys Glu His Asp Phe Gln Met Leu
Phe Ala Pro Cys Cys His Gln Cys Gly Glu Phe
Ile Ile Gly Arg Val Ile Lys Ala Met Asn Asn
Ser Trp His Pro Glu Cys Phe Arg Cys Asp Leu
Cys Gln Glu Val Leu Ala Asp Ile Gly Phe Val
Lys Asn Ala Gly Arg His Leu Cys Arg Pro Cys
His Asn Arg Glu Lys Ala Arg Gly Leu Gly Lys
Tyr Ile Cys Met Thr Gly Ser Asn Met Ser Asp
Ala Leu Ala Asn Ala Val Cys Gln Arg Cys Gln
Ala Arg Phe Ser Pro Ala Glu Arg Ile Val Asn
Ser Asn Gly Glu Leu Tyr His Glu His Cys Phe
Val Cys Ala Gln Cys Phe Arg Pro Phe Pro Glu
Gly Leu Phe Tyr Glu Phe Glu Gly Arg Lys Tyr
Cys Glu His Asp Phe Gln Met Leu Phe Ala Pro
Cys Cys Gly Ser Cys Gly Glu Phe Ile Ile Gly
Arg Val Ile Lys Ala Met Asn Asn Asn Trp His
Pro Gly Cys Phe Arg Cys Glu Leu Cys Asp Val
Glu Leu Ala Asp Leu Gly Phe Val Lys Asn Ala
Gly Arg His Leu Cys Arg Pro Cys His Asn Arg
Glu Lys Ala Lys Gly Leu Gly Lys Tyr Ile Cys
Gln Arg Cys His Leu Val Ile Asp Glu Gln Pro
Leu Met Phe Arg Ser Asp Ala Tyr His Pro Asp
His Phe Asn Cys Thr His Cys Gly Lys Glu Leu
Thr Ala Glu Ala Arg Glu Leu Lys Gly Glu Leu
Tyr Cys Leu Pro Cys His Asp Lys Met Gly Val
Pro Ile Cys Gly Ala Cys Arg Arg Pro Ile Glu
Gly Arg Val Val Asn Ala Leu Gly Lys Gln Trp
His Val Glu His Phe Val Cys Ala Lys Cys Glu
Lys Pro Phe Leu Gly His Arg His Tyr Glu Lys
Lys Gly Leu Ala Tyr Cys Glu Thr His Tyr Asn
Gln Leu Phe Gly Asp Val Cys Tyr Asn Cys Ser
His Val Ile Glu Gly Asp Val Val Ser Ala Leu
Asn Lys Ala Trp Cys Val Ser Cys Phe Ser Cys
Ser Thr Cys Asn Ser Lys Leu Thr Leu Lys Asp
Lys Phe Val Glu Phe Asp Met Lys Pro Val Cys
Lys Arg Cys Tyr Glu Lys Phe Pro Leu Glu Leu
Lys Lys Arg Leu Lys Lys Leu Ser Glu Leu Thr
Ser Arg Lys Ala Gln Pro Lys Ala Thr Asp Leu
Asn Ser Ala.

In contrast to normal seronegative controls, PINCH is robustly expressed in the brains and CSF of HIV-infected individuals. Specifically, PINCH was detected in the neuronal nucleus, cytoplasm, and processes and in the extracellular matrix (Rearden et al., Journal of Neuroscience Research 86:2535-2542 (2008)). PINCH distribution patterns differed between HIVE patients and HIV patients with no reported CNS alterations (Id.).

For a review of PINCH function, see Kovalevich et al., J. Cell. Physiol. 226: 940-947 (2011).

What is needed is a method to assess the severity of disease in neurodegenerative disorders having an associated Tau component.

### SUMMARY OF THE INVENTION

A method for determining disease severity in a subject afflicted with a tauopathy-related neurodegenerative disease comprises:

detecting the level of a PINCH protein in a test sample comprising brain tissue or cerebrospinal fluid from a subject afflicted with a tauopathy-related neurodegenerative disease;

comparing said level of said PINCH protein in said test sample with the level of said PINCH protein in at least one control sample;

wherein the level of said PINCH protein in the test sample as compared to the level in the control sample indicates the relative severity of the tauopathy-related neurodegenerative disease afflicting the subject. For example, an elevated level of PINCH protein is an indication of tauopathy-related neurodegenerative disease severity.

In some embodiments, the control sample comprises a sample from a normal subject. In other embodiments, the control sample comprises a sample from a subject afflicted with a tauopathy-related neurodegenerative disease. In some embodiments, the control sample comprises a reference sample of known PINCH protein level.

In some embodiments, the level of the PINCH protein in the test sample is compared with the level of the PINCH protein in a panel of control samples comprising varying levels of the PINCH protein, and determining the severity of the tauopathy-related neurodegenerative disease in the subject from a comparison of the PINCH protein level in the test sample and control samples.

Also provided is a method of monitoring the progression of a tauopathy-related neurodegenerative disease in a subject. The method comprises: obtaining a first test sample comprising brain tissue or cerebrospinal fluid from a subject afflicted with a tauopathy-related neurodegenerative disease at a first time point and a second test sample comprising brain tissue or cerebrospinal fluid from said subject at a second time point; determining the level of PINCH protein from said first and second test samples; and comparing the level of said PINCH protein determined in said first test sample to the level of said PINCH protein from said second test sample, wherein an elevated level, decreased or unchanged level of said PINCH protein in said second test sample relative to the level of said PINCH protein in said first sample is an indication that the a tauopathy-related neurodegenerative disease has worsened, diminished or remained unchanged in said subject.

In some embodiments of the aforesaid methods, the PINCH protein comprises total PINCH protein. In other embodiments, the PINCH protein comprises insoluble PINCH protein. In other embodiments, the PINCH protein comprises a PINCH protein isoform. In certain embodiments, the PINCH isoform is a post-translationally modified PINCH protein. In some embodiments, the PINCH isoform has a molecular weight of about 37 kDa. In other embodiments, the PINCH protein has a molecular weight of about 42, about 51 or about 71 kDa, depending on its modification and/or association with other molecules.

In some embodiments of the aforesaid methods, the PINCH protein is PINCH-1. In some embodiments of the aforesaid methods, the PINCH protein is PINCH-2. In embodiments, the PINCH protein may comprise total PINCH-1, insoluble PINCH-1 or an isoform of PINCH-1. In some embodiments of the aforesaid methods, the PINCH protein is PINCH-2. In embodiments, the PINCH protein may comprise total PINCH-2, insoluble PINCH-2 or an isoform of PINCH-2.

Examples of tauopathy-related neurodegenerative diseases according to the above methods include Alzheimer's Disease (AD), frontotemporal dementia (FTD), HIV encephalitis (HIVE), CTE, TBI or seizure.

In a related invention, a method for determining disease severity in a subject afflicted with a disease condition is provided. The method comprises: detecting the level of a PINCH protein isoform in a test sample from a subject afflicted with a disease; comparing said level of said PINCH protein isoform in said test sample with the level of said PINCH protein isoform in at least one control sample; wherein the level of said PINCH protein isoform in the test sample as compared to the level in the control sample indicates the relative severity of the disease condition afflicting the subject. For example, an elevated level of PINCH protein is an indication of disease severity. Representative disease conditions include, for example, neurodegenerative diseases, multiple sclerosis, cancer, epilepsy, renal failure, cardiomyopathy and tumorigenesis. In some embodiments, the PINCH isoform is a post-translationally modified PINCH protein. In some embodiments, the PINCH isoform has a molecular weight of about 37 kDa.

In some embodiments, the PINCH protein isoform is an isoform of PINCH-1. In other embodiments, the PINCH protein isoform is an isoform of PINCH-2.

In certain embodiments of the aforementioned methods, the level of PINCH protein is determined by enzyme-linked immunosorbent assay (ELISA), Western Blot analysis, immunoprecipitation, immunofluorescent assay, radioimmunoassay, chemiluminescent assay, flow cytometry, immunocytochemistry, mass spectrometry, two-dimensional electrophoresis, or any combination thereof.

In any of the aforementioned embodiments of the invention, where the subject is undergoing treatment for tauopathy-related neurodegenerative disease or other disorder, or is a candidate for such treatment, the treatment may be initiated, adjusted or ceased according to the outcome of PINCH protein or PINCH isoform level determination. For example, treatment may be initiated, accelerated or enhanced upon a finding that the tauopathy-related neurodegenerative disease afflicting the subject is severe, where the disease severity has increased from a prior determination, or where the disease severity has been the same since a prior determination. Likewise, treatment may be deferred, attenuated or halted in response to a PINCH level determination indicating less severe disease, or a PINCH level determination indicating that the severity of the disease has lessened since a prior determination.

### BRIEF DESCRIPTION OF THE DRAWINGS

FIGS. 1A and 1B show the levels of soluble hp-Tau (s262) and PINCH in AD brains from different disease stages of AD and HIVE compared to age-matched normal control brains. AD Br1 is least severe, AD Br3 is moderately severe and AD Br5 is most severe. Total Tau is a marker used for comparison of levels of hpTau relative to overall levels of Tau, with bars in FIG. 1B indicated more hpTau in more severe disease. GAPDH in FIG. 1A is a marker for even protein loading.

FIG. 1C shows the immunoprecipitation of proteins from a representative AD brain tissue sample (AD Br3) with anti-PINCH antibody and Western analyses with anti-AT8 (an hp-Tau-binding antibody), confirming the direct interaction of PINCH with hp-Tau.

FIGS. 1D-1H show the results of a double immunofluorescence labeling of brain tissue from a representative AD patient, demonstrating PINCH co-localization (arrowheads and arrows) with hp-Tau (FIG. 1D, 1E), CHIP (FIG. 1F), and Hsp70 (FIG. 1G) but not with Hsp90 (FIG. 1H). FIG. 1D shows single overlay of nuclei (FIG.D.1), PINCH (FIG.D.2), hpTau (FIG.D.3) and overlay (FIG.D.4).

FIGS. 1I and 1J show the results of a double immunofluorescence labeling of brain tissue from a representative HIVE patient, demonstrating PINCH co-localization (arrowheads) with hp-Tau.

FIG. 2A is Western analysis for PINCH and hpTau of brain tissue from the human Tau transgenic mouse, P310S (TauTg) compared to brain tissue from a wild type normal mouse (nonTg). Analyses of the anterior frontal cortex (Ant-FC), ventro-lateral posterior cortex (V-L-post-FC), posterior frontal cortex (Post-FC), cerebellum from the Tau-Tg mouse and nonTg wildtype control mouse indicated increased soluble hp-Tau and PINCH in all regions of the Tau-Tg mouse brain. GAPDH is a marker for even protein loading.

FIGS. 2B and 2C show the results of a double immunolabeling of hippocampal tissue of the non-transgenic control mouse (FIG. 2B) and the P310S human Tau transgenic mouse (FIG. 2C). The results show that PINCH and hp-Tau are detected in the human Tau transgenic mouse and appear to co-localize in the neurons (FIG. 2C). Immunoreactivity was not detected for either PINCH or hp-Tau in the control wild type mouse (FIG. 2B).

FIG. 3A shows the level of PINCH, hp-Tau and total Tau in brain tissues from a normal control case, 3 AD cases (AD Br1, AD Br3, AD Br5), an HIVE and an FTD case. The buffers RAB (most soluble), RIPA (less soluble) and formic acid (FA, least soluble) separate proteins based on solubility. These results show that in disease, hpTau loses solubility, which is a hallmark of disease and that PINCH also loses solubility and levels increase. FIG. 3B shows a stained gel to illustrate the amount of total protein in each sample for even loading.

FIG. 4A shows PINCH immunoreactivity in a series of spots (dark arrows) at approximately 37 kDa in the CSF from HIV patients (HIV+) that are absent in the CSF of control patients. These experiments were conducted in triplicate on the CSF from nine different control and nine different HIV patients. Light arrows represent non-specific IgG reaction.

FIG. 4B shows the same gel as FIG. 4A immunoreacted with hpTau antibody indicting increased hpTau in the HIV+ patients CSF and less or absent reactivity in the controls.

FIG. 4C shows representative control and HIV CSF samples immunoreacted with PINCH and hpTau antibodies indicating co-existence in common sample.

FIG. 4D illustrates the concept that as disease worsens, PINCH levels increase. These samples are from the same patient collected at different stages of HIV disease. Clearly on the high HIV (reflecting high levels of virus in the CSF) image more PINCH is present than on low HIV (reflecting low levels of HIV in the CSF).

FIG. 5 shows a western blot from the CSF of seven control patients and 2 patients that suffered from traumatic brain injury (TBI). PINCH levels are robustly increased in the TBI patients.

FIGS. 6A-6C show western blots from brain tissue from 4 controls and 7 epilepsy patients. FIG. 6A shows increased PINCH in the epilepsy tissue. FIGS. 6B and 6C show increased hpTau in epilepsy tissues compared to controls.

FIGS. 7A-7D show reciprocal immunoprecipitation (IP) of human CSF from an HIV patient. FIGS. 7A and 7B show IP with PINCH antibody, followed by Western blot with hpTau antibody. FIG. 7A is a lighter exposure of FIG. 7B. FIGS. 7C and 7D show IP with hpTau antibody, and Western blot with PINCH antibody. FIG. 7C is a lighter exposure of FIG. 7D. These results confirm PINCH/hpTau binding.

### DEFINITIONS

As used herein, each of the following terms has the meaning associated with it in this section.

As used herein, each of the following terms has the meaning associated with it in this section.

The articles “a” and “an” are used herein to refer to one or to more than one (i.e. to at least one) of the grammatical object of the article. By way of example, “an element” means one element or more than one elements.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

By “tauopathy” is meant a class of neurodegenerative diseases associated with the pathological aggregation of tau protein in the brain. By “tauopathy-related neurodegenerative disease” is meant a neurodegenerative disease or disorder in which a pathological aggregation of tau protein in the brain.

As used herein, “severity of a tauopathy-related neurodegenerative disease” refers generally to the extent of the pathological aggregation of tau protein and associated symptoms of neurological impairment.

As used herein, the term “subject” or “patient” refers to any animal (e.g., a mammal) including, but not limited to, humans and non-human primates, afflicted with a tauopathy-related neurodegenerative disease. Typically, the terms “subject” and “patient” are used interchangeably herein in reference to a human subject.

As used herein, a “normal subject” or “control subject” refers to a subject that does not manifest clinical symptoms of neurodegenerative disorder.

As used herein, a “normal reference” refers to a normal subject or to a population of normal subjects.

By “PINCH protein” is meant to include, unless indicated to the contrary, either PINCH-1 and PINCH-2, including all isoforms and/or post-translationally modified forms thereof, including complexes or associations comprising PINCH protein with one or more other molecules.

“Sample” or “test sample” as used herein means a biological material isolated from an individual. The test sample may contain any biological material suitable for detecting the desired biomarkers, and may comprise cellular and/or non-cellular material obtained from the individual. The sample or test sample may comprise, unless indicated otherwise, a tissue, a biological fluid, blood, plasma or serum.

As used herein, a “detector molecule” is a molecule that may be used to detect a compound of interest. Non-limiting examples of a detector molecule are molecules that bind specifically to a compound of interest, such as, but not limited to, an antibody, a cognate receptor or binding partner, an aptamer, and a small molecule.

By the term “specifically binds,” as used herein with respect to a detector molecule such as an antibody, is meant a detector molecule that recognizes a specific binding partner, such as an antigen, but does not substantially recognize or bind other molecules in a sample.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, an “immunoassay”, “western analyses”, “immunoreactivity”, “immunoreacted”, “immunoblot”, refers to any binding assay that uses an antibody capable of binding specifically to a target molecule to detect and quantify the target molecule.

As used herein, “post-translational modification” refers to any chemical modification of a polypeptide after it is produced. Commonly, a post-translational modification involves attaching at least one moiety to the polypeptide chain, however, post-translational modification can be cleavage of the polypeptide chain, proteolytic processing, the formation of disulfide bonds, and the like. Non-limiting examples of post-translational modifications include glycosylation, phosphorylation, acylation, acetylation, methylation, sulfonation, prenylation, isoprenylation, ubiquitination, biotinylation, formylation, citrullination, myristolation, ribosylation, sumoylation, gamma carboxylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, demethylation, formation of covalent cross-links, formation of cystine, formation of pyroglutamate, CPI anchor formation, hydroxylation, iodination, methylation, nitrosylation, oxidation, proteolytic processing, racemization, selenoylation, sulfation, transfer-RNA mediated addition of amino acids to proteins such as arginylation, and the like. See, for instance, Proteins—Structure and Molecular Properties, 2^{nd} Ed., T. E. Creighton, W.H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al, (1990) Analysis for Protein Modifications and Nonprotein Cofactors, Methods Enzymol. 182:626-46 and Rattan et al. (1992) Protein Synthesis: Posttranslational Modifications and Aging, Ann. NY Acad. Sci. 663:48-62.

It is understood that any and all whole or partial integers between any ranges set forth herein are included herein. As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### Abbreviations

AD: Alzheimer's Disease.

CSF: Cerebrospinal fluid.

CNS: Central nervous system.

CTE: Chronic traumatic encephalopathy

FTD: Frontotemporal dementia.

HIVE: HIV encephalitis.

hp-Tau or hpTau: Hyperphosphorylated Tau.

HSR: The heat shock response.

IR: Immunoreactive.

NFTs: Neurofibrillary tangles

TBI: traumatic brain injury.

### DETAILED DESCRIPTION OF THE INVENTION

According to the present invention, it has been discovered that PINCH binds to hp-Tau and may contribute to neuropathology associated with hpTau. It is demonstrated that during the cellular stress response, PINCH binds to hp-Tau and may contribute to changes in intracellular levels and subcellular localization of hp-Tau. These studies address a new mechanism by which AD and HIV-associated neurocognitive disorders may intersect. Without wishing to be bound by any theory, it is hypothesized that in diseases with a tauopathy component, PINCH is expressed by neurons to influence cell survival through its interactions with Tau, the heat shock protein response machinery and other cellular components.

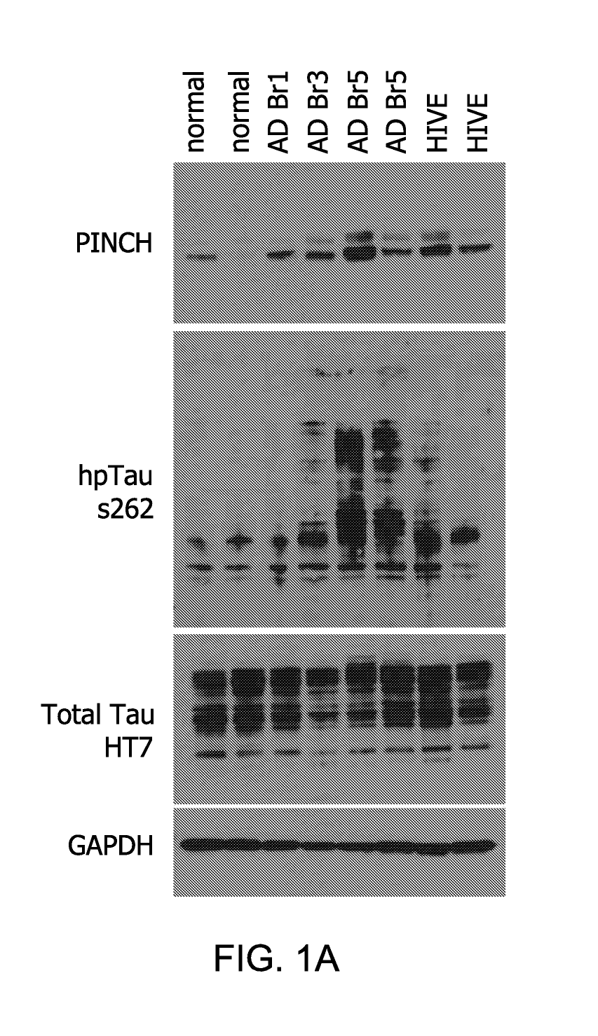

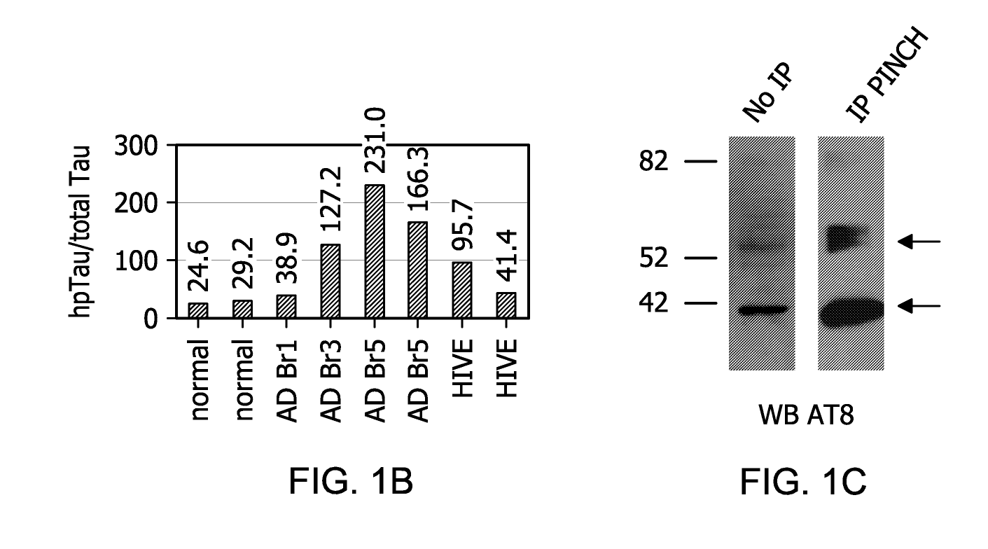

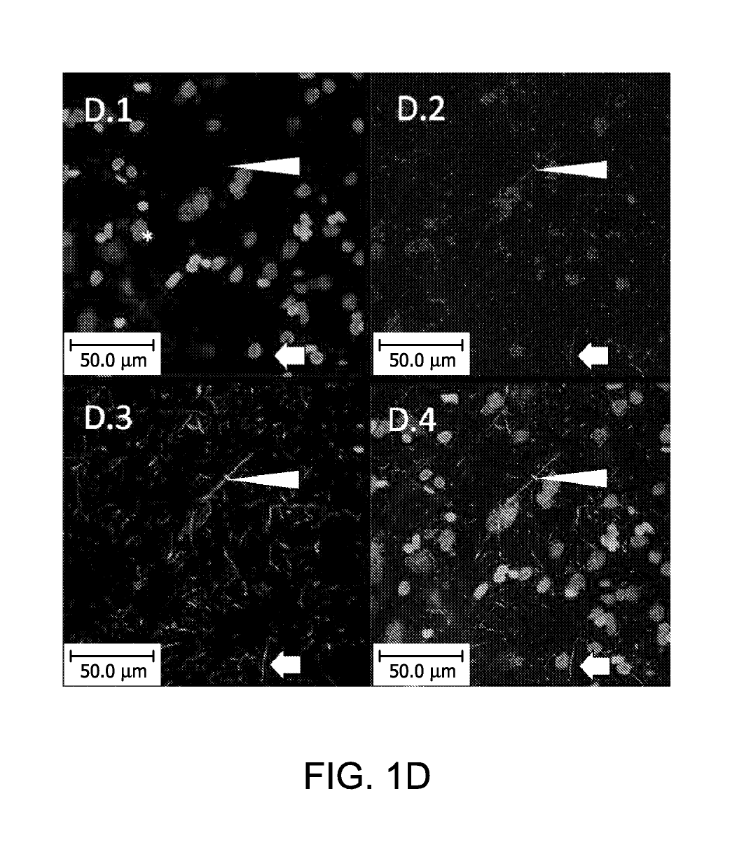

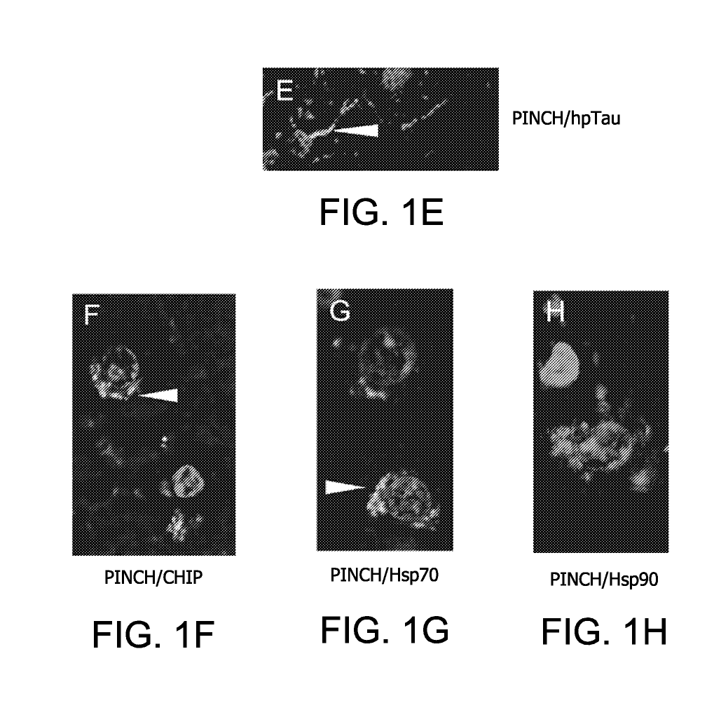

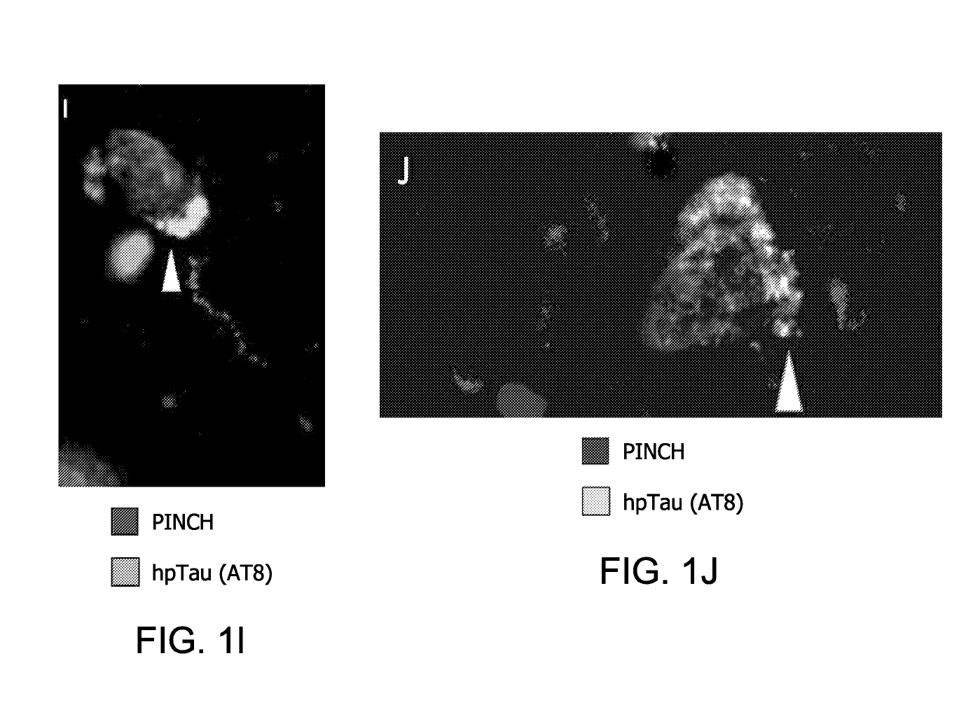

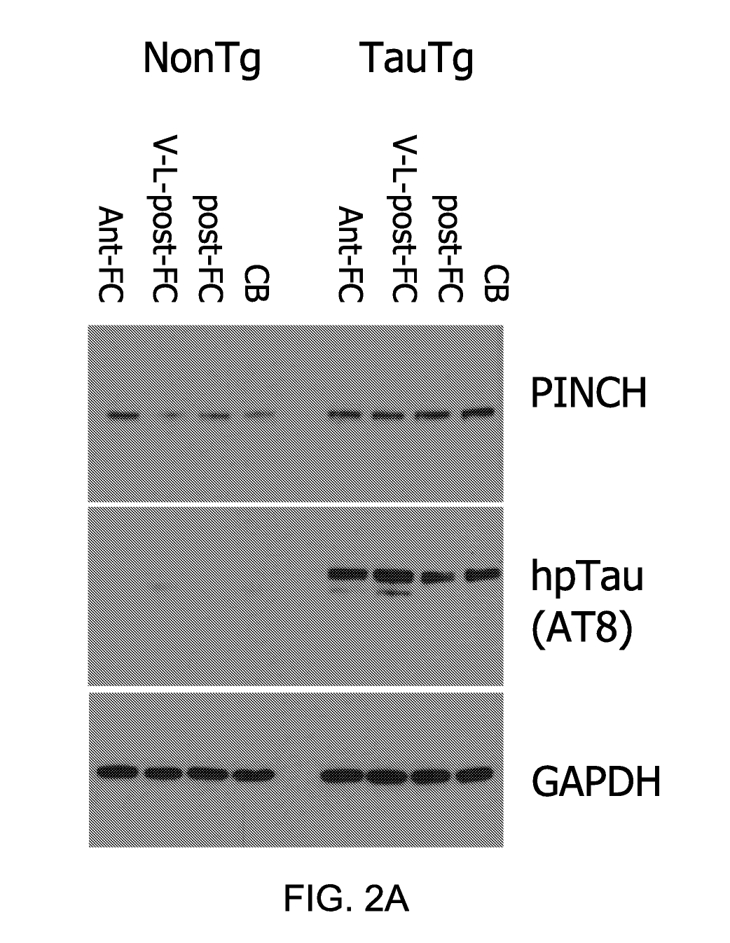

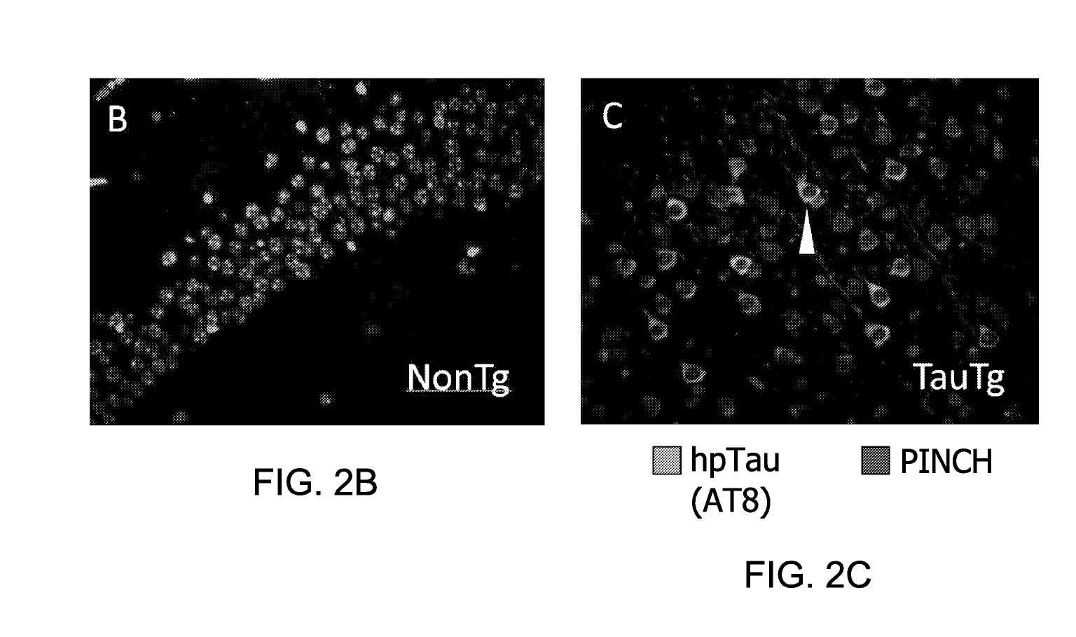

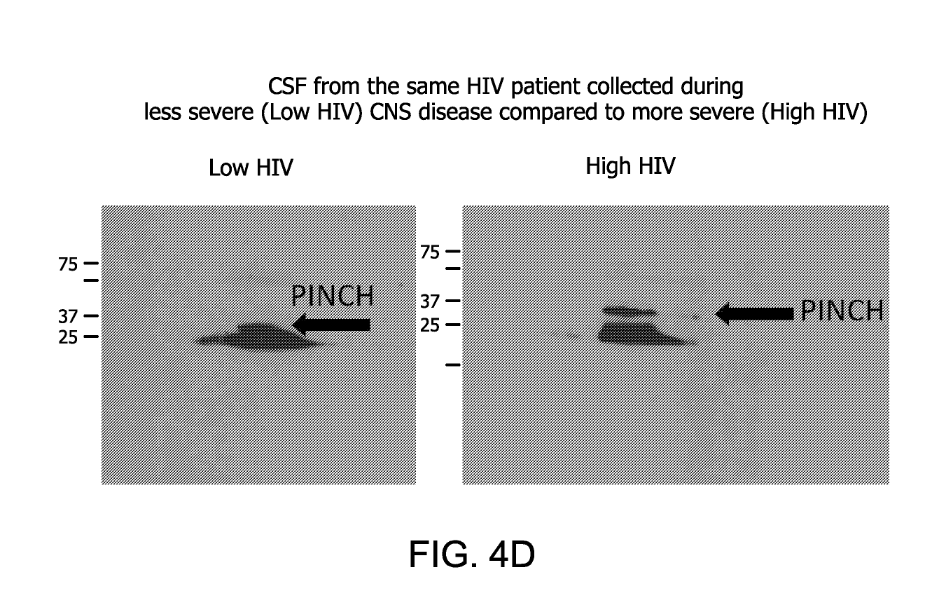

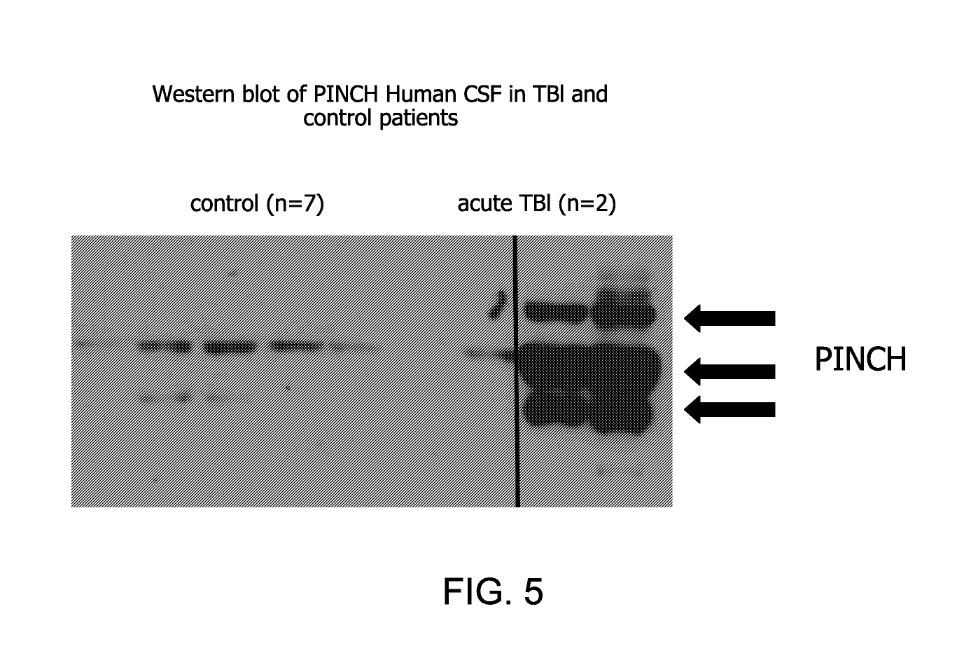

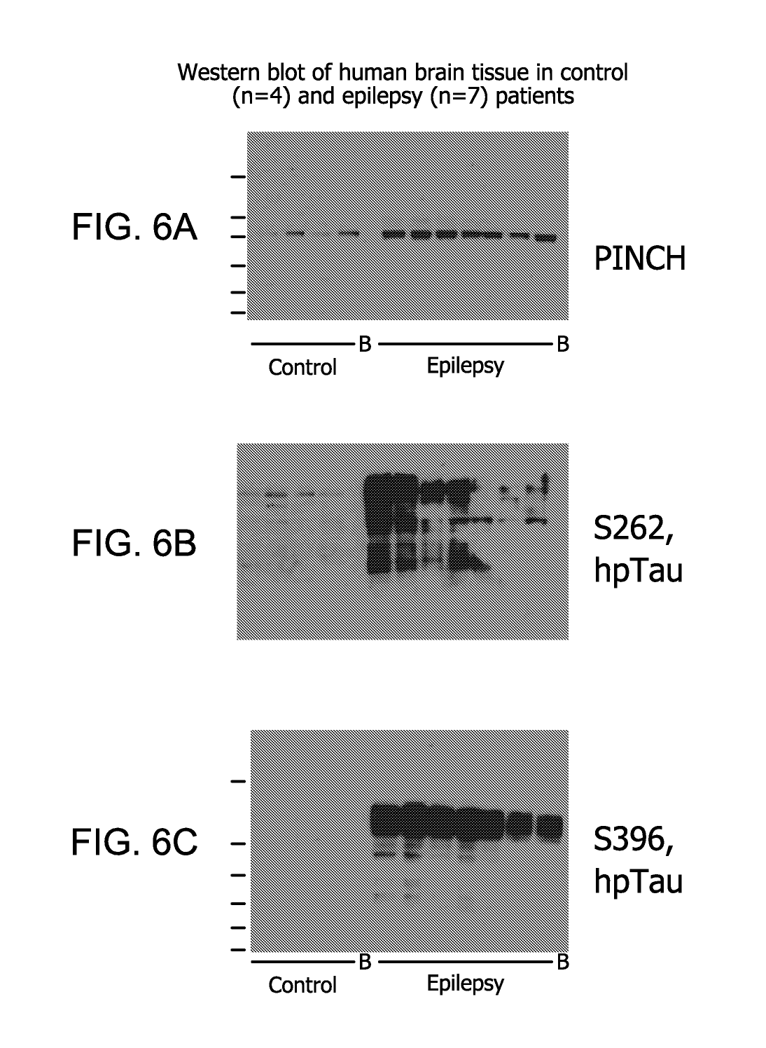

As demonstrated hereinafter, hp-Tau accumulation is accompanied by increased PINCH expression. Accordingly, in one embodiment of the invention, total PINCH proteins in the brain tissue or CSF of patients suffering from neurodegenerative diseases with a tauopathy component are used to assess disease severity. As shown hereinafter, while significantly increased levels of soluble hp-Tau in AD, HIVE brains compared to age-matched control brains were detected (FIGS. 1A, 1B), significantly greater levels of PINCH accompanied increased hp-Tau in AD and HIVE patients' brains compared to controls (FIGS. 1A, 1B). Increased levels of PINCH were also detected in the CSF from TBI patients compared to control (FIG. 5). In brain tissue from epilepsy patients compared to control greater levels of PINCH (FIG. 6A) were accompanied by increased hpTau (FIGS. 6B and 6C). PINCH expression is increased in the brain of the P310S human Tau transgenic mouse (Tau-Tg) (FIG. 2A). Increased soluble hp-Tau and PINCH is present in all regions of the Tau-Tg mouse brain (FIG. 2A: anterior frontal cortex (Ant-FC), ventro-lateral posterior cortex (V-L-post-FC), posterior frontal cortex (Post-FC) and cerebellum), but not in wild type non-transgenic control mice (FIG. 2A).

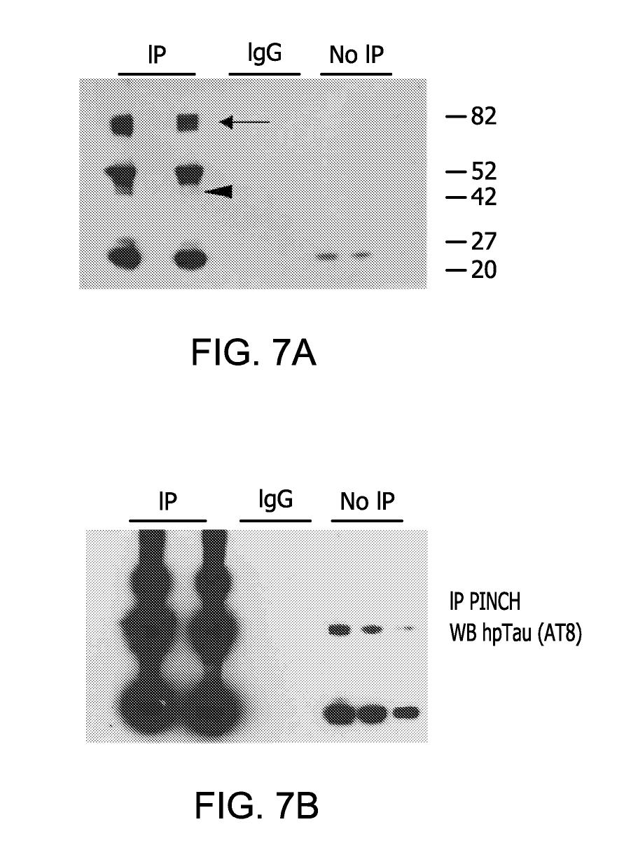

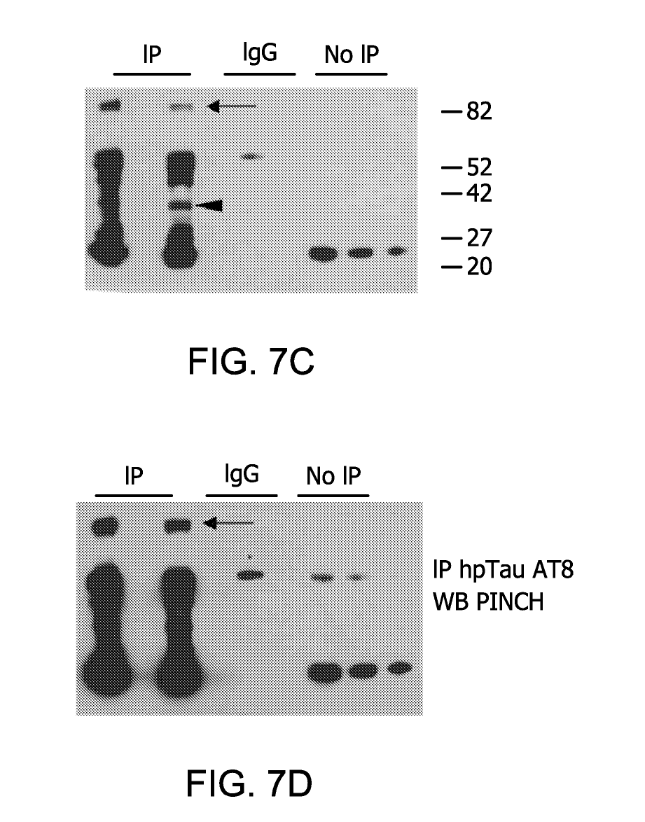

Immunoprecipitation of proteins from AD brain tissue with anti-PINCH antibody and Western analyses with hp-Tau specific anti-AT8 antibody demonstrated that PINCH interacts directly with hp-Tau (FIG. 1C, arrows). Likewise, reciprocal immunoprecipitation of proteins from an HIV patient with antibody against PINCH and western analyses with hp-Tau antibody (FIGS. 7A, 7B) and vice versa (FIGS. 7C, 7D), indicate that PINCH and hp-Tau interact. Further evidence of PINCH/hp-Tau interaction is provided by their co-localized in neurons in the brain of a representative AD patient (FIGS. 1D, 1E, arrows and arrowheads), an HIV patient (FIG. 1I, 1J) and the Tau transgenic mouse (FIG. 2C, arrowhead). PINCH and human Tau are not detectable in the wild type non-transgenic mouse brain (FIG. 2B).

Thus, hp-Tau accumulation, a hallmark of tauopathy-related neurodegenerative disease and a marker for disease severity, is itself accompanied by increased PINCH expression. The level of PINCH is accordingly a marker for tauopathy-related neurodegenerative disease severity.

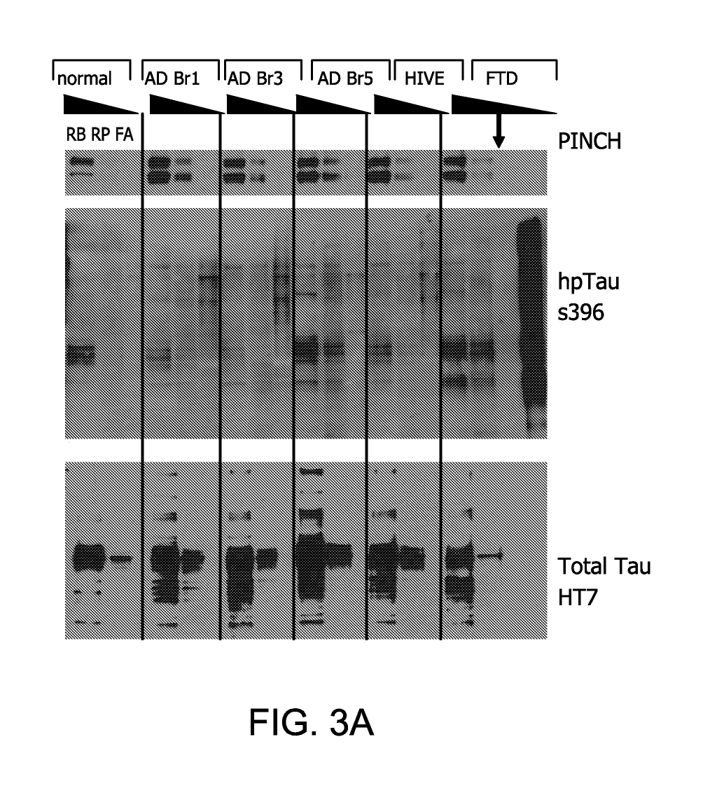

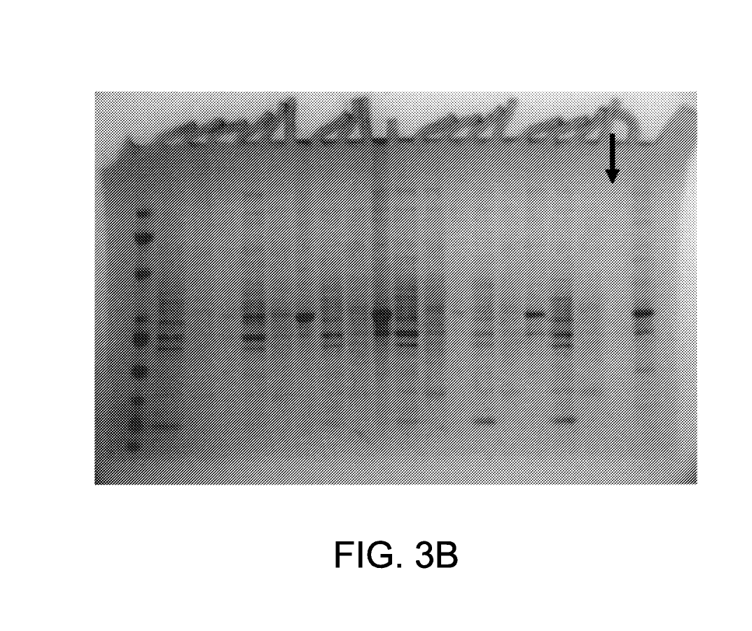

As demonstrated hereinafter, in patients suffering from neurodegenerative diseases with a tauopathy component, PINCH is increased, binds to hp-Tau and accompanies Tau as it loses solubility in disease progression. Accordingly, the level of insoluble PINCH in patient samples is indicative of disease severity. As shown in FIG. 3A, analysis of brain tissue from a normal control case demonstrated low levels of PINCH in a RAB (RB) preparation containing soluble proteins (FIG. 3A). Low levels of hp-Tau were also detected in the RAB (RB) fraction; the majority of total Tau is present in the highly soluble RAB fraction (FIG. 3A). Most of the phosphorylated Tau species in patients without tauopathy is highly soluble.

In contrast, patients suffering from AD, HIVE and FTD, are characterized by increased levels of PINCH and hp-Tau (FIG. 3A). The AD Br1 case, which was diagnosed as mild, showed increased PINCH present in the most soluble RAB (RB) and the less soluble RIPA (RP) fraction; however, most of the total Tau is present in the RAB (RB) soluble fraction as expected (FIG. 3A). In the more severe AD cases (Br3, 5) and in HIVE, more hpTau is detected in the RP (less soluble) and FA (insoluble) fractions. In the FTD case, PINCH levels increased as observed in the AD and HIVE cases. In the FTD case, a formic acid soluble fraction, FA (which represents insoluble proteins and such preparation is required to detect insoluble proteins), most hpTau is present. Accordingly, the level of less soluble PINCH in patient samples is indicative of disease severity.

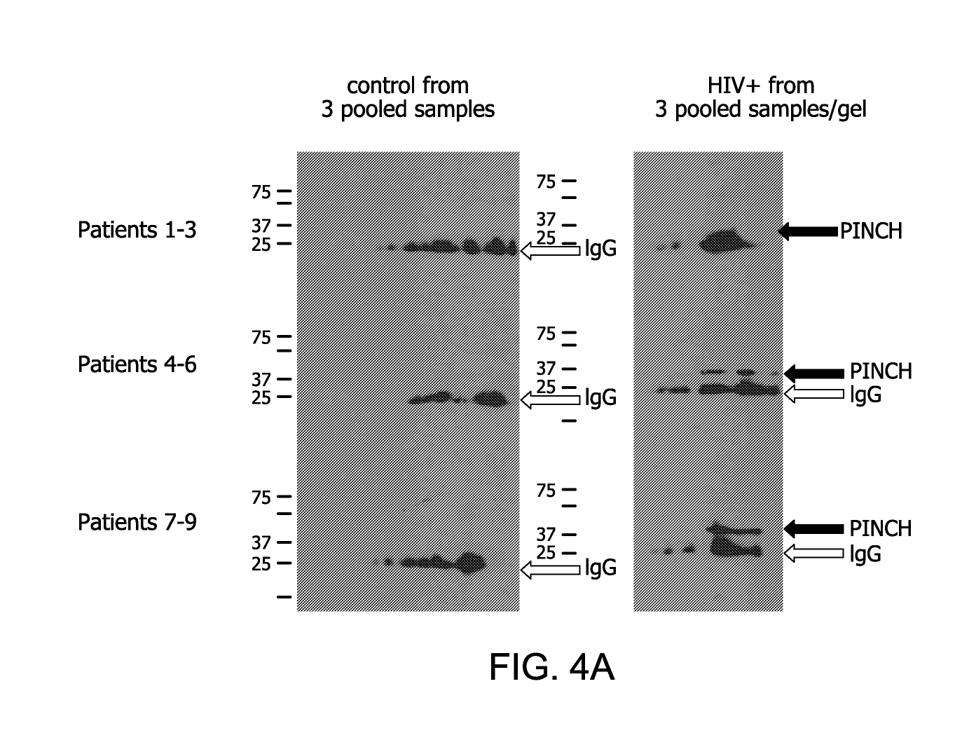

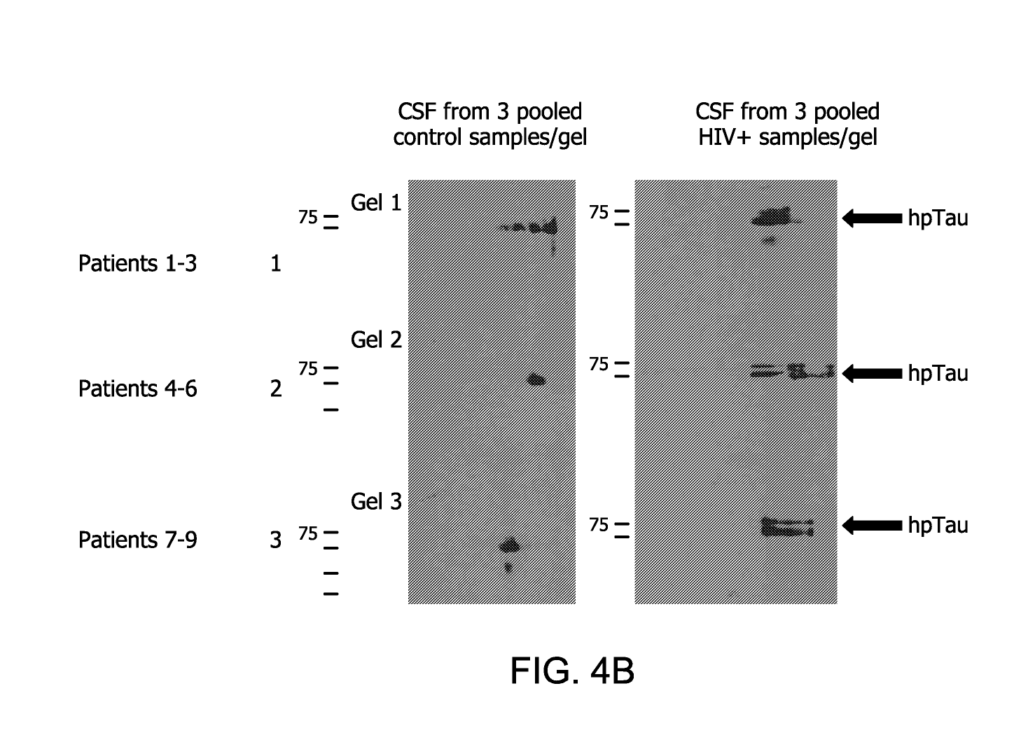

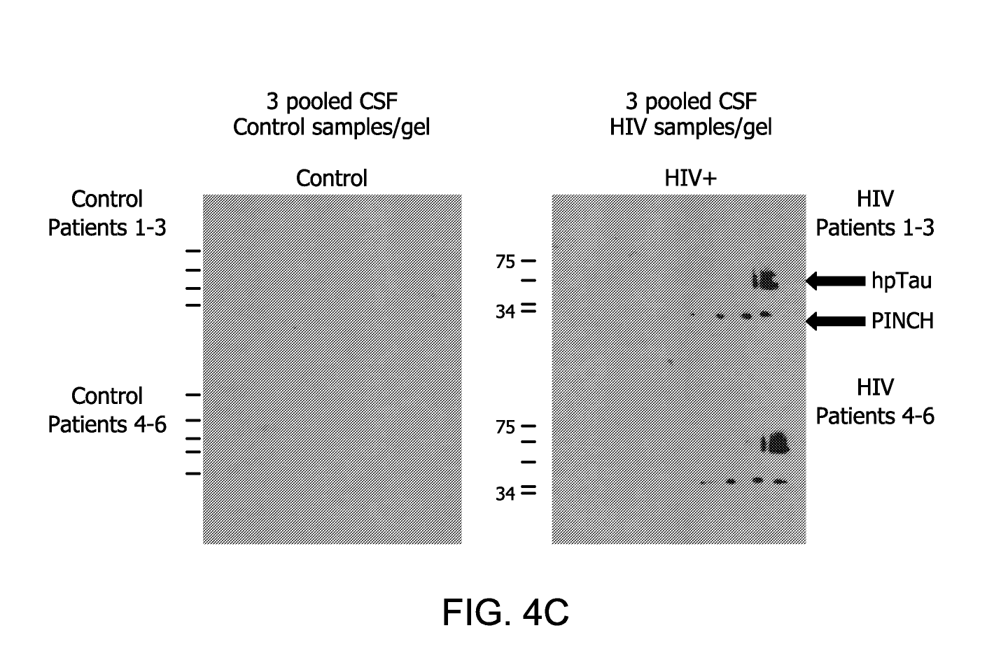

In another embodiment of the invention, the detection of one or more PINCH isoforms is a marker for the detection of disease. See, e.g. FIGS. 4A, 4C. PINCH isoforms are identifiable, for example, by two dimensional (2D) gel electrophoresis and mass spectroscopy methods. Identifiable PINCH isoforms may comprise, for example, PINCH molecules that may contain post-translational modifications or comprise species of proteins with phosphate, acetyl, sumo, glycosyl or other alterations. Without wishing to be bound by any theory, these types of changes may play significant roles in PINCH's ability to interact with other proteins and ultimately influence cell fate.

Detection of different PINCH protein isoforms in the brain tissue, CSF or blood of patients may be used to determine disease severity in patients suffering from a variety of diseases including but not limited to neurodegenerative diseases, multiple sclerosis, cancer, epilepsy, renal failure, cardiomyopathy and tumorigenesis. As illustrated by FIG. 4D (arrow), in CSF samples from the same patient at different stages of disease as defined by a higher HIV load in the CSF (High HIV) reflecting more severe disease, more PINCH is detected with different isoforms present in the severe disease stage compared to low HIV in less severe disease. This data illustrates that as disease worsens in a given patient, increased PINCH levels and different isoforms of PINCH can be detected in the CSF of this patient.

In one embodiment, the PINCH isoform, which is a marker for disease severity has a molecular weight of about 37 kDa upon 2-D gel electrophoresis (FIG. 4), and is detected in CSF of HIV patients. In HIVE patients' CSF multiple hp-Tau IR spots are detected that are not observed in the control patients' CSF (FIGS. 4B, 4C, arrows).

The methods described herein rely on assessing the level of total PINCH, the level of insoluble PINCH, and/or the level of a PINCH isoform. The level of total PINCH and/or insoluble PINCH correlates with the severity of a tauopathy-related neurodegenerative disease. The sample may be a brain tissue sample or a CSF sample. The level of PINCH isoform is determined to assess the severity of at least one of the following: neurodegenerative disease (including but not limited to tauopathy-related neurodegenerative disease); multiple sclerosis; cancer; epilepsy; renal failure; cardiomyopathy; and tumorigenesis. For determine the level of PINCH isoform, the sample may comprise any relevant bodily tissue or fluid, including without limitation, brain tissue, CSF, peripheral whole blood, and components thereof such as blood serum (“serum”) and blood plasma (“plasma”). The sample is obtained from the subject using conventional methods in the art. For instance, one skilled in the art knows how to draw blood and how to process it in order to obtain serum and/or plasma for use in practicing the described methods. Generally speaking, the method of obtaining and storing, if necessary, the sample preferably maintains the integrity of the one or more biomarkers the disclosed herein such that it can be accurately quantified in the biological fluid sample.

The methods of the invention include quantitatively measuring the level of total PINCH, the level of insoluble PINCH, or the level of a PINCH isoform (collectively, “PINCH protein”). In some embodiments, the PINCH protein is a PINCH isoform comprising a post-translationally modified PINCH protein. Methods of quantitatively assessing the level of an unmodified or post-translationally modified protein in a biological fluid are well known in the art, and are applicable to PINCH proteins. In some embodiments, assessing the level of an unmodified or post-translationally modified protein involves the use of a detector molecule for the biomarker. In preferred embodiments, the detector molecule is specific for either the unmodified or the post-translationally modified protein biomarker. Detector molecules can be obtained from commercial vendors or can be prepared using conventional methods in the art. Exemplary detector molecules include, but are not limited to, an antibody that binds specifically to the unmodified or post-translationally modified biomarker, a naturally-occurring cognate receptor, or functional domain thereof, for the unmodified or post-translationally modified biomarker, an aptamer that binds specifically to the unmodified or post-translationally modified biomarker, and a small molecule that binds specifically to the unmodified or post-translationally modified biomarker. Small molecules that bind specifically to an unmodified or post-translationally modified biomarker can be identified using conventional methods in the art, for instance, screening of compounds using combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. Methods for preparing aptamers are also well-known in the art.

The methods of the invention also include detecting the presence or absence of an isoform comprising a post-translational modification, using detector molecules that are specific for a certain post-translationally modified biomarker.

In a preferred embodiment, the level of PINCH protein is assessed using an antibody. Thus, exemplary methods for assessing the level of PINCH protein in a biological fluid sample include various immunoassays, for example, immunohistochemistry assays, immunocytochemistry assays, ELISA, capture ELISA, sandwich assays, enzyme immunoassay, radioimmunoassay, fluorescence immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY. Solid phase immunoassays can be particularly useful. Where two or more PINCH proteins are assessed, a panel of antibodies in an array format can be utilized. Custom antibody microarrays or chips can be obtained commercially.

Antibodies can be used in various immunoassay-based protein determination methods such as Western blot analysis, immunoprecipitation, radioimmunoassay (RIA), immunofluorescent assay, chemiluminescent assay, flow cytometry, immunocytochemistry and enzyme-linked immunosorbent assay (ELISA).

In an enzyme-linked immunosorbent assay (ELISA), an enzyme such as, but not limited to, horseradish peroxidase (HRP), alkaline phosphatase (AP), beta-galactosidase or urease can be linked, for example, to an antigen antibody or to a secondary antibody for use in a method of the invention. A horseradish-peroxidase detection system may be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which may be used with the chromogenic substrate p-nitrophenyl phosphate to yield a soluble product readily detectable at 405 nm. Similarly, a beta-galactosidase detection system may be used with the chromogenic substrate o-nitrophenyl-beta-D-galactopyranoside (ONPG) to yield a soluble product detectable at 410 nm. Alternatively, a urease detection system may be used with a substrate such as urea-bromocresol purple (Sigma Immunochemicals, St. Louis, Mo.). Useful enzyme-linked primary and secondary antibodies can be obtained from any number of commercial sources.

For chemiluminescence and fluorescence assays, chemiluminescent and fluorescent secondary antibodies may be obtained from any number of commercial sources. Fluorescent detection is also useful for detecting antigen or for determining a level of antigen in a method of the invention. Useful fluorochromes include, but are not limited to, DAPI, fluorescein, Hoechst 33258, R-phycocyanin, B-phycoerythrin, R-phycoerythrin, rhodamine, Texas red and lissamine- Fluorescein- or rhodamine-labeled antigen-specific antibodies.

Radioimmunoassays (RIAs) are described for example in Brophy et al. (1990, Biochem. Biophys. Res. Comm. 167:898-903) and Guechot et al. (1996, Clin. Chem. 42:558-563). Radioimmunoassays are performed, for example, using Iodine-125-labeled primary or secondary antibody.

Western blotting may also be used to detect and or determine the level of phosphorylated Cdc27. Western blots may be quantified using well known methods such as scanning densitometry (Parra et al., 1998, J. Vasc. Surg. 28:669-675).

A signal emitted from a detectable antibody is analyzed, for example, using a spectrophotometer to detect color from a chromogenic substrate; a radiation counter to detect radiation, such as a gamma counter for detection of Iodine-125; or a fluorometer to detect fluorescence in the presence of light of a certain wavelength. Where an enzyme-linked assay is used, quantitative analysis of the amount of antigen is performed using a spectrophotometer. It is understood that the assays of the invention can be performed manually or, if desired, can be automated and that the signal emitted from multiple samples can be detected simultaneously in many systems available commercially. Antigen-antibody binding can also be detected, for example, by mass spectrometry.

The antibody used to detect a PINCH protein in a sample in an immunnoassay can comprise a polyclonal or monoclonal antibody. The antibody can comprise an intact antibody, or antibody fragments capable of specifically binding antigen. Such fragments include, but are not limited to, Fab and F(ab′)₂ fragments.

Techniques for detecting and quantifying (such as with respect to a control) antibody binding are well-known in the art. Antibody binding a PINCH protein may be detected through the use of chemical reagents that generate a detectable signal that corresponds to the level of antibody binding and, accordingly, to the level of marker protein expression. Examples of such detectable substances include enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵1, ¹³¹1, ³⁵S, or ³H.

Antibody binding may be detected through the use of a secondary antibody that is conjugated to a detectable label. Examples of detectable labels include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Preferred enzymes of particular interest include horseradish peroxidase (HRP) and alkaline phosphatase (AP).

PINCH protein can be detected and quantified by aptamer-based assays, which are very similar to antibody-based assays, but with the use of an aptamer instead of an antibody. An “aptamer-based” assay is thus an assay for the determination of polypeptide that relies on specific binding of an aptamer. An aptamer can be any polynucleotide, generally an RNA or a DNA, which has a useful biological activity in terms of biochemical activity or molecular recognition attributes. Usually, an aptamer has a molecular activity such as having an enzymatic activity or binding to a polypeptide at a specific region (i.e., similar to an epitope for an antibody) of the polypeptide. It is generally known in the art that an aptamer can be made by in vitro selection methods. In vitro selection methods include a well-known method called systematic evolution of ligands by exponential enrichment (a.k.a. SELEX). Briefly, in vitro selection involves screening a pool of random polynucleotides for a particular polynucleotide that binds to a biomolecule, such as a polypeptide, or has a particular activity that is selectable. Generally, the particular polynucleotide represents a very small fraction of the pool therefore, a round of amplification, usually via polymerase chain reaction, is employed to increase the representation of potentially useful aptamers. Successive rounds of selection and amplification are employed to exponentially increase the abundance of a particular aptamer. In vitro selection is described in Famulok, M.; Szostak, J. W., In Vitro Selection of Specific Ligand Binding Nucleic Acids, Angew. Chem. 1992, 104, 1001. (Angew. Chem. Int. Ed. Engl. 1992, 31, 979-988.); Famulok, M.; Szostak, J. W., Selection of Functional RNA and DNA Molecules from Randomized Sequences, Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX; Mol. Biol. Reports 1994, 20, 97-107; and Burgstaller, P.; Famulok, M. Synthetic ribozymes and the first deoxyribozyme; Angew. Chem. 1995, 107, 1303-1306 (Angew. Chem. Int. Ed. Engl. 1995, 34, 1189-1192), U.S. Pat. No. 6,287,765, U.S. Pat. No. 6,180,348, U.S. Pat. No. 6,001,570, U.S. Pat. No. 5,861,588, U.S. Pat. No. 5,567,588, U.S. Pat. No. 5,475,096, and U.S. Pat. No. 5,270,163, which are incorporated herein by reference.

Substantially pure PINCH protein, which can be used as an immunogen for raising polyclonal or monoclonal antibodies, or as a substrate for selecting aptamers, can be prepared, for example, by recombinant DNA methods. For example, the cDNA of the marker protein can be cloned into an expression vector by techniques within the skill in the art. An expression vector comprising sequences encoding the maker protein can then be transfected into an appropriate eukaryotic host, whereupon the protein is expressed. The expressed protein can then be isolated by any suitable technique.

In one embodiment, detection of a PINCH protein is carried out using an immunoblot method that relies on electrophoretic separation of proteins from a sample and detection with a specific antibody.

The generation of polyclonal antibodies is accomplished by inoculating the desired animal with an antigen and isolating antibodies, which specifically bind the antigen therefrom.

Monoclonal antibodies directed against a PINCH protein may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against a biomarker such as H3.3 can be generated, for instance, from mice immunized with the biomarker using standard procedures as referenced herein.

For use in preparing an antibody, PINCH protein may be purified from a biological source that endogenously comprises the protein, or from a biological source recombinantly-engineered to produce or over-produce the protein, using conventional methods known in the art. The amino acid sequence, and exemplary nucleic acid sequences, for human and other mammalian PINCH-1 and PINCH-2 are readily available in public sequence databases, such as National Library of Medicine's genetic sequence database GenBank® (Benson et al., 2008, Nucleic Acids Research, 36(Database issue):D25-30).

Nucleic acid encoding the monoclonal antibody obtained using the procedures described herein may be cloned and sequenced using technology which is available in the art, and is described, for example, in Wright et al. (1992, Critical Rev. Immunol. 12(3,4):125-168) and the references cited therein.

Other methods for assessing the level of a PINCH protein include chromatography (e.g., HPLC, gas chromatography, liquid chromatography), capillary electrophoresis and mass spectrometry (e.g., MS, MS-MS). For instance, a chromatography medium comprising a cognate receptor for a PINCH protein, an aptamer that binds specifically to the protein, or a small molecule that binds specifically to the protein can be used to substantially isolate the PINCH protein from the sample of tissue or biological fluid.

The level of substantially isolated PINCH protein can be quantified directly or indirectly using a conventional technique in the art such as spectrometry, Bradford protein assay, Lowry protein assay, biuret protein assay, or bicinchoninic acid protein assay, as well as immunodetection methods.

The level of a PINCH protein in a biological sample can be normalized. For instance, the level can be normalized to another component of the sample, whose level is independent of whether the patient suffers from tauopathy-related neurodegenerative disease. It is well within the skill of the skilled artisan to select a suitable component for normalization.

The practice of the invention is illustrated by the following non-limiting example. The invention should not be construed to be limited solely to the compositions and methods described herein, but should be construed to include other compositions and methods as well. One of skill in the art will know that other compositions and methods are available to perform the procedures described herein.

### Example 1

### Measurement of PINCH and Tau in Brain Tissue and Cerebrospinal Fluid by Western Analysis

Accumulation of hp-Tau is a hallmark of numerous neurodegenerative diseases including AD (Lee, et al. (2011) Cold Spring Harbor Perspectives in Medicine 1(1), a006437) and FTD (Yoshiyama, et al. (2002) J Biol Chem 277(41), 38328-38338); and is reported in HIVE (Anthony et al. (2006) Acta Neuropathol 111(6), 529-538; Patrick, et al. (2011) Am J Pathol 178(4), 1646-1661). The levels of soluble hp-Tau and PINCH in post-mortem brain tissue from AD, HIVE, FTD (FIG. 1), and resections from epilepsy patients (FIG. 6) were assessed as follows. CSF was collected either post-mortem or after injury in TBI cases.

One hundred milligrams of frozen tissues from grey matter of frontal (AD, HIVE, FTD) or temporal (epilepsy) cortex was homogenized on ice in HEPES buffer (1 mM HEPES, 5 mM benzamidine, 2 mM 2-mercaptoethanol, 3 mM EDTA, 0.5 mM magnesium sulfate, 0.05% sodium azide, 1 mM sodium orthovanadate, and 0.01 mg/ml leupeptin). Protein concentrations were determined by the bicinchoninic acid assay (BCA Protein Assay Kit; Pierce Rockford, Ill.) following the manufacturer's protocol, and 25 μg of protein was loaded per well. For CSF, constant volumes of 3 μl CSF were loaded per well. Proteins were separated by electrophoresis for 1 hr at 200 V on 4-12% Bis-Tris NuPage Gels (Invitrogen, Carlsbad, Calif.). Primary antibodies were used at 1:1000 for Western analyses unless otherwise indicated: PINCH1 (BD, Rockville, Md.), PINCH2 (Zhang, et al. (2002) J Biol Chem 277(41), 38328-38338)), Hsp90 (Abcam, Cambridge, Mass.), Hsp70 (1:5000) (Abcam), CHIP (1:500) (Abcam), tubulin (1:2000) (Sigma), GAPDH (1:5000) (SCBT, Santa Cruz, Calif.), Grb-2 (Cell Signaling, Danvers, Mass.). Anti-Tau antibodies from Thermo-Fischer (Pittsburgh, Pa.) included: HT7 against human total Tau; AT8 and AT100 against PHF-Tau. Anti-Tau phospho-S262 and -S396 were from Abeam (Cambridge, Mass.). Enhanced chemiluminescence was detected with the Western Lightning Chemiluminescence Reagent Plus Kit (PerkinElmer Life Sciences, Boston, Mass.) and recorded using the Bio-Rad VersaDoc Imaging System model 3000 (Bio-Rad, Hercules, Calif.).

For immunoprecipitations (IP), CSF was mixed 4:1 vol:vol with lysis buffer (50 mM Tris-HCl, 150 mM NaCl, 1% NP-40, protease inhibitor cocktail) and incubated 30 min on ice. The mixture was incubated with 2.5 μg of antibody (either AT8 for hp-Tau antibody or PINCH antibody) overnight at 4° C. After incubation, 20 μl of Protein-A bead slurry was added and the samples were rotated end over end mix for 4 hours at 4° C. The beads with protein conjugates were washed 5 times with 500 μl of lysis buffer. After centrifugation and removal of supernatant, 50 μl of 1× Laemmli sample buffer was added to bead pellet. The sample was heated to 100° C., centrifuged and the proteins were analyzed by Western blotting.

Immunofluorescent labeling of formalin-fixed, paraffin-embedded frontal cortex brain tissues from HIV, AD, and control patients was conducted on serial sections. Four month-old male mice (Tau-Tg and wild type) were euthanized, brains were removed and one hemisphere was fixed in formalin for immunolabeling. Five or 40 μm serial sections from the formalin-fixed paraffin-embedded tissues were processed in citrate buffer for antigen retrieval and rehydrated through ethanol to water. Sections were blocked with normal human serum, incubated with the primary antibodies: anti-PINCH (1:200), anti-Tau AT8 (1:200), anti-CHIP (1:100), Hsp-70 (1:200), and Hsp-90 (1:200) overnight in a humidified chamber at room temperature, rinsed three times with PBS, then incubated with fluorescein isothiocyanate (FITC)-conjugated secondary antibody (1:500) or Texas-red isothiocyanate (TRITC)-tagged secondary antibodies (1:200) (Thermo-Scientific) for 2 h at room temperature in the dark. After washing with PBS, the sections were re-blocked and incubated overnight at room temperature in a humidified chamber with the second primary antibody. After washing, sections were incubated with the second secondary antibody for 1 h at room temperature in the dark. Finally, sections were cover-slipped with an aqueous based mounting media containing DAPI for nuclear labeling (Vector Laboratories), visualized with a Nikon ultraviolet inverted microscope, and processed with deconvolution software (Slidebook 4.0, Intelligent Imaging, Denver, Colo.). Deconvolution was performed using SlideBook4 software, allowing acquisition of multiple 0.2 mm thick digital sections and 3-D reconstruction of the image. Confocal microscopy was conducted on 40 μm sections using the Leica EL6000, with LAF AS software (Leica Microsystems, Buffalo Grove, Ill., USA).

### Results and Significance:

Increased levels of soluble hp-Tau in AD (5-8 fold greater), and HIVE (2-4 fold greater) brains compared to age-matched control brains were detected (FIGS. 1A, 1B). Immunoprecipitation of proteins from AD brain tissue with anti-PINCH antibody and Western analyses with anti-AT8 antibody confirmed the interaction of PINCH with hp-Tau (FIG. 1C, arrows). Immunoprecipitation of protein from the CSF of a representative HIV patient with anti-PINCH antibody and western analyses with anti-hpTau antibody (FIGS. 7A, 7B) and the reciprocal (IP with anti-hpTau antibody and western analyses with anti-PINCH antibody (FIGS. 7C, 7D) confirms the interaction of hpTau and PINCH proteins.

Since our in vitro data also showed that PINCH interacts with heat shock factors, double immunofluorescence labeling of brain tissue from a representative AD patient was conducted. Results showed PINCH co-localization with hp-Tau, CHIP, and Hsp70, but not with Hsp90 (FIGS. 1I, 1J, arrowheads).

Similar experiments in the brains of HIVE patients showed increased levels of soluble hp-Tau compared to an age-matched control (FIG. 1A), as previously reported (Anthony et al. (2006) Acta Neuropathol 111(6), 529-538; Patrick, et al. (2011) Am J Pathol 178(4), 1646-1661)). PINCH and hp-Tau also co-localized in the brain of a representative HIVE patient (FIGS. 1I, 1J, arrowheads).

Further support for the interaction of hp-Tau and PINCH in diseases with a pathological component of hp-Tau was shown as follows by evaluating brain tissue from the human Tau transgenic mouse, P310S, by Western analyses. Western analyses of different brain regions from the Tau-Tg mouse and control mouse indicated increased soluble hp-Tau and PINCH in all regions (FIG. 2A: anterior frontal cortex (Ant-FC), ventro-lateral posterior cortex (V-L-post-FC), posterior frontal cortex (Post-FC), cerebellum). Likewise, double immunolabeling of hippocampal tissue indicated that in the P310S human Tau transgenic mouse, PINCH and hp-Tau were detected and co-localized (FIG. 2C); whereas, in the control, immunoreactivity was not detected for either protein (FIG. 2B). As observed in AD and HIVE patients' brains, PINCH and hp-Tau appeared to co-localize in the neurons of the Tau transgenic mouse (FIG. 3C, arrowhead). The results with respect to hp-Tau are consistent with reports that hp-Tau levels increase in AD, HIVE and in the human-Tau transgenic mouse. Shown here for the first time is that hp-Tau accumulation is accompanied by increased PINCH expression. Thus, the results indicate a role of PINCH in neuropathological processes in tauopathy associated with AD and HIVE, and that PINCH expression may also serve as a marker for disease severity and progression in tauopathy-related neurodegenerative disease.

### Example 2

### Changes in PINCH Levels and Solubility Correlate with Disease Severity in Neurodegenerative Diseases with a Tauopathy Component

Upon hyperphosphorylation of Tau, the accumulation of paired helical filaments and the formation of tangles are accompanied by increased hp-Tau insolubility. To determine the expression levels and changes in solubility of PINCH during loss of Tau solubility in disease, brain tissues from normal control, AD, HIVE and FTD patients were processed to separate proteins into different fractions based on solubility, as follows.

Proteins of different solubility were extracted from brain in buffers of increasing stringency, using a slightly modified protocol previously described (Ke, et al. (2009) PloS one 4(11), e7917). Briefly, frozen brain tissue from the gray matter of frontal cortex was weighed and 100 mg was homogenized in 10 μl/mg RAB buffer (100 mM 2-(N-morpholino) ethanesulphonic acid (MES; pH 7.0), 1 mM EDTA, 0.5 mM MgSO₄, 750 mM NaCl, 20 mM NaF, 1 mM Na₃VO₄ and complete protease inhibitors (Sigma)) with a plastic pestle in 1.5 mL tubes. The homogenate was passed through a 29 G insulin needle (Terumo, USA), incubated on ice for 30 min and centrifuged at 50,000×g for 20 min at 4° C. The RAB-soluble proteins in the supernatant were collected. The pellet was resuspended in 7.5 μl/mg RIPA buffer (50 mM Tris (pH 8.0), 150 mM NaCl, 1% NP40, 5 mM EDTA, 0.5% sodium deoxycholate, 0.1% sodium dodecyl sulfate) and centrifuged at 50,000×g for 20 min at 4° C. The supernatant containing RIPA-soluble proteins was collected. The pellet was resuspended in 7.5 μl/mg 70% formic acid (FA) in distilled water. The samples were incubated for 30 min on ice and centrifuged at 50,000×g for 20 min at 4° C. The supernatants containing FA-soluble proteins (also considered RIPA insoluble proteins) were collected. The FA fractions were dialyzed against PBS overnight at 4° C., and an equal volume of 50 mM Tris-HCl (pH 7.4) was added to each sample. Protein concentrations were determined by the standard Bradford assay and equal amounts of protein were loaded per well. For CSF, 600 μl of fluid were collected and processed as described above to assess levels of PINCH and Tau of different solubility.

### Results and Significance:

In brain tissue from a normal control case, low levels of PINCH were detected in the RAB (RB) preparation containing soluble proteins (FIG. 3A). Low levels of hp-Tau were also detected predominantly in the RAB (RB) fraction, with the majority of total Tau present in the highly soluble RAB (RB) fraction (FIG. 3A) confirming that most of the phosphorylated Tau species in patients without tauopathy is highly soluble. In patients suffering from AD, HIVE and FTD, increased levels of PINCH and hp-Tau were observed (FIG. 3A). The AD Br1 case, which was diagnosed as mild, showed increased PINCH present in the RAB soluble and the less soluble RIPA fraction however, most of the total Tau is present in the RAB soluble fraction (FIG. 3A). In the most severe AD case (AD Br5) more hpTau was present in the RIPA and FA fractions with more PINCH in the RIPA fraction. In the HIVE and FTD cases, PINCH levels increased dramatically in the RAB and RIPA fractions (FIG. 3A). The significance of the two PINCH immunoreactive (IR) bands is not currently known, but may represent changes in post-translational modifications of the PINCH protein, but may reflect different isoforms. These results show that in patients suffering from neurodegenerative diseases with a tauopathy component, PINCH is increased, binds to hp-Tau and may accompany Tau as it loses solubility in disease progression.

### Example 3

### Detection of PINCH Isoforms

Unlike traditional one-dimensional electrophoresis, 2D gel electrophoresis separates protein based not only on molecular weight in the vertical dimension, but also by charge or isoelectric focusing point in the horizontal dimension. This technology allows for the identification of changes such as post-translational modifications that may represent different isoforms or species of proteins with phosphate, acetyl, sumo, glycosyl or other alterations. Importantly, these types of changes may play significant roles in a protein's ability to interact with other proteins and ultimately influence cell fate. Two dimensional gel electrophoresis was used to detect PINCH isoforms as follows.

CSF was mixed with isoelectric focusing lysis solution containing 7 M urea, 2 M thiourea, 4% CHAPS, 100 mM DTT, and protease inhibitor cocktail at 4:1 vol:vol ratio. The first-dimension isoelectric focusing was carried out on an Amersham Biosciences, Inc. Mulyiphor II system essentially as described by the manufacturer. Pre-cast immobilized pH gradient strips (18 cm; pH 3-10 NL) were used for the first-dimensional separation for a total focusing time of 25 kV-h. The strips were equilibrated with a solution containing 6 M urea, 30% glycerol, 2% SDS, 50 mM Tris (pH 8.8) reduced with 100 mm DTT and directly applied to a 15% isocratic SDS-polyacrylamide gel electrophoresis (PAGE) overnight at 60-mA constant current. Proteins are transferred on to nitrocellulose membranes and standard Western blot analyses are conducted with anti-PINCH and anti-hp-Tau antibodies. For further characterization of isoforms, gels are stained with SPYRO red, imaged using Z3 software and individual spots are selected for further analyses. Selected spots are removed from the gel, transferred to a 96-well microtiter plate for trypsin digestion followed by protein identification by standard peptide mass fingerprinting and Matrix-Assisted Laser Desorption/Ionization/Time-of-Flight (MADLDI/TOF) mass spectroscopy.

### Results and Significance:

In this context, FIGS. 4A, 4C and 4D show PINCH immunoreactive (IR) as a series of spots (dark arrows) at approximately 37 kDa in the CSF from HIV patients. These spots are absent in the CSF of control patients. Moreover, each of the 3-4 spots represents a corresponding protein with a different isoelectric point (or pH). Of note, there is a series of IR spots at approximately 26 kDa that are present in both control and HIV patients' CSF (light arrows). These are non-specific IgG bands. These experiments were conducted in triplicate on the CSF from 9 different control and 9 HIV patients where the CSF from 3 patients was pooled in each representative 2D immunoblot.

Changes in PINCH isoforms accompany worsening disease. In FIG. 4D the CSF from the same patient taken at two different times 30 days apart was analyzed by 2D analyses. The High HIV represents more severe disease when the patient had higher viral loads in the CSF. The low HIV represents a less severe stage of disease when CSF levels of virus were low. Not only is there more PINCH in the High HIV blot (arrow), but different isoforms (spots) are detected when compared to the low HIV.

As expected, in HIVE patients' CSF multiple hp-Tau IR spots are detected that are not observed in the control patients' CSF (FIGS. 4B, 4C, arrrows). These results alone are suggestive of not only increased hp-Tau, but also of multiple isoforms of Tau in disease.

Particular PINCH isoform(s) will interact with one or more hp-Tau isoforms and will be useful for determining if disease has worsened, diminished or remained unchanged in numerous Tauopathy diseases.

### Example 4

### Mass Spectroscopy Identification of Post-Translational Modifications of 2D Gel Electrophoresis of PINCH Isoforms

The following is a representative protocol for identification and quantification of PINCH isoforms in test samples, coupling 2D gel electrophoresis (2D) and MALDI/TOF mass spectroscopy. The analysis is performed to determine the amount of PINCH isoforms that are specifically post-translationally modified in disease. In brief, the first dimension of separation is isoelectric focusing (IEF), which uses narrow range IPG strips (pI 4-7 and 6-10). Each sample is run in triplicate. The second dimension of separation is SDS-PAGE. Proteins in the 2DE gel are revealed by staining with SYPRO-Ruby fluorescent total protein stain (Molecular Probes, Eugene, Oreg.). Fluorescence images are captured and analyzed, and individual spot volumes are calculated by density/area integration and normalized for slight difference in protein loading across gels using PDQuest (Boden & Merali (2011) Methods Enzymol 489: 67-82; Kelsen et al. (2008) Am J Respir Cell Mol Biol 38: 541-50). Protein spots are excised from the 2DE gel and subjected to tryptic digestion as described previously (Boden & Merali 2011, Kelsen et al. 2008).

Isoforms detected by 2D gel are analyzed using the following approaches. The desalted tryptic peptides are dried in a vacuum centrifuge and re-solubilized in 30 L of 0.1% (vol/vol) trifluoroacetic acid. The tryptic peptide sample is loaded onto a 2 microgram capacity peptide trap (CapTrap™; Michrom Bioresources, Auburn, Calif.), separated by a C18 capillary column (15 cm 75 Agilent) at 300 nl/min delivered by an Agilent 1100 LC pump. A mobile-phase gradient is run using mobile phase A (1% acetonitrile/0.1% formic acid) and B (80% acetonitrile/0.1% formic acid) from 0 to 10 min with 0-15% B followed by 10-60 min with 15-60% B and 60-65 min with 60-100% B.

Nanoelectrospray ionization (ESI) tandem mass spectroscopy (MS) is performed using a Brukers HCT Ultra ion trap mass spectrometer. ESI is delivered using a distal-coating spray Silica tip (ID 20 μM, tip inner ID 10 μM, New Objective) at a spray voltage of −1300 V. Using automatic switching between MS and MS/MS modes, MS/MS fragmentation is performed on the two most abundant ions on each spectrum using collision-induced dissociation with active exclusion (excluded after two spectra, and released after 2 min). The complete system is fully controlled by HyStar 3.1 software.

Mass spectra processing is performed using Brukers Biotools (Version 2.3.0.0) with search and quantitation toolbox options. The generated de-isotoped peak list is submitted to a Mascot server 2.2 and searched against the Swiss-Prot database (version 56.6 of 16 Dec. 2008, 405506 sequences). Mascot search parameters are set as follows: Homo sapiens (20413 sequences); enzyme, trypsin with maximal 3 missed cleavage sites with variable modification: Acetyl (K), Acetyl (Protein N-term), Carbamidomethyl (C), Methyl (C-term), Methyl (DE), Dimethyl (RK) Oxidation (M), Phospho (ST), Phospho (Y); 0.60 Da mass tolerance for precursor peptide ions; and 0.9 Da for MS/MS fragment ions. All peptide matches are filtered using an ion score cutoff of 10. Only unique peptides with scores ≧35 (p<0.05) are confidently assigned. In each MS/MS spectrum, a total of at least four b- and y-ions are observed. These criteria are used to search against a reversed decoy Swiss-Prot database, to obtain false positive match. For added stringency, proteins with scores above 40 are used for comparisons between samples.

The disclosures of each and every patent, patent application, publication and GenBank record cited herein are hereby incorporated herein by reference in their entirety.

One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

What is claimed is:

1. A method for determining disease severity in a subject afflicted with a tauopathy-related neurodegenerative disease comprising:
detecting the level of a PINCH protein in a test sample comprising brain tissue or cerebrospinal fluid from a subject afflicted with a tauopathy-related neurodegenerative disease;
comparing said level of said PINCH protein in said test sample with the level of said PINCH protein in at least one control sample;
wherein the level of said PINCH protein in the test sample as compared to the level in the control sample indicates the relative severity of the tauopathy-related neurodegenerative disease afflicting the subject.

2. The method according to claim 1, wherein the control sample comprises a sample from a normal subject.

3. The method according to claim 1, wherein the control sample comprises a sample from a subject afflicted with a tauopathy-related neurodegenerative disease.

4. The method according to claim 1, wherein the control sample comprises a reference sample of known PINCH protein level.

5. The method according to claim 1, wherein the level of said PINCH protein in said test sample is compared with the level of said PINCH protein in a panel of control samples comprising varying levels of said PINCH protein, and determining the severity of the tauopathy-related neurodegenerative disease in said subject from a comparison of said PINCH protein level in the test sample and control samples.

6. A method of monitoring the progression of a tauopathy-related neurodegenerative disease in a subject, said method comprising:
obtaining a first test sample comprising brain tissue or cerebrospinal fluid from a subject afflicted with a tauopathy-related neurodegenerative disease at a first time point and a second test sample comprising brain tissue or cerebrospinal fluid from said subject at a second time point;
detecting the level of a PINCH protein from said first and second test samples; and
comparing the level of said PINCH protein determined in said first test sample to the level of said PINCH protein from said second test sample, wherein an elevated level, decreased or unchanged level of said PINCH protein in said second test sample relative to the level of said PINCH protein in said first sample is an indication that the a tauopathy-related neurodegenerative disease has worsened, diminished or remained unchanged in said subject.

7. The method according to claim 1, wherein the PINCH protein comprises total PINCH protein.

8. The method according to claim 1, wherein the PINCH protein comprises insoluble PINCH protein.

9. The method according to claim 1, wherein the PINCH protein comprises a PINCH protein isoform.

10. The method according to claim 9, wherein the PINCH isoform is a post-translationally modified PINCH protein.

11. The method according to claim 10, wherein the PINCH protein isoform has a molecular weight of about 37 kDA.

12. The method according to claim 1, wherein determining the levels of PINCH protein is by enzyme-linked immunosorbent assay (ELISA), Western Blot analysis, immunoprecipitation, immunofluorescent assay, radioimmunoassay, chemiluminescent assay, flow cytometry, immunocytochemistry, mass spectrometry, two dimensional electrophoresis, or any combination thereof.

13. The method according to claim 1 wherein the test sample comprises brain tissue or cerebrospinal fluid.

14. (canceled)

15. The method according to claim 1 wherein the tauopathy-related neurodegenerative disease is Alzheimer's Disease, frontotemporal dementia or HIV encephalitis.

16. (canceled)

17. (canceled)

18. A method for determining disease severity in a subject afflicted with a disease condition comprising:
detecting the level of a PINCH protein isoform in a test sample from a subject afflicted with a disease;
comparing said level of said PINCH protein isoform in said test sample with the level of said PINCH protein isoform in at least one control sample;
wherein the level of said PINCH protein isoform in the test sample as compared to the level in the control sample indicates the relative severity of the disease condition afflicting the subject.

19. The method according to claim 18, wherein the disease condition is selected from the group consisting of neurodegenerative diseases, multiple sclerosis, cancer, epilepsy, renal failure, cardiomyopathy and tumorigenesis.

20. The method according to claim 18, wherein the PINCH isoform is a post-translationally modified PINCH protein.

21. The method according to claim 18, wherein the PINCH isoform has a molecular weight of about 37 kDA.

22. The method according to claim 18, wherein determining the level of PINCH protein is by enzyme-linked immunosorbent assay (ELISA), Western Blot analysis, immunoprecipitation, immunofluorescent assay, radioimmunoassay, chemiluminescent assay, flow cytometry, immunocytochemistry, mass spectrometry, two dimensional electrophoresis, or any combination thereof.

23. The method according to claim 9 wherein detecting said PINCH protein isoform comprises subjecting said test sample to two dimensional electrophoresis and isoelectric focusing to separate PINCH protein isoforms in said sample.

24. The method according to claim 23, wherein the level of said PINCH protein isoform(s) is determined by MALDI/TOF mass spectroscopy.

25. The method according to claim 18 wherein detecting said PINCH protein isoform comprises subjecting said test sample to two dimensional electrophoresis and isoelectric focusing to separate PINCH protein isoforms in said sample.

26. The method according to claim 25, wherein the level of said PINCH protein isoform(s) is determined by MALDI/TOF mass spectroscopy.
